(19) United States
(12) Patent Application Publication
(10) Pub. No.: US 2026/0210903 A1
(43) Pub. Date: Jul. 23, 2026

(54) Organic Electrolyte-Gated Field Effect Transistor Biosensor

(71) Applicant: Carleton University, Ottawa, Ontario (CA)

(72) Inventors: Ravi PRAKASH, Ottawa, Ontario (CA); Roslyn S. MASSEY, Gloucester, Ontario (CA)

(21) Appl. No.: 19/112,623

(22) PCT Filed: Jul. 25, 2024
(86) PCT No.: PCT/CA2024/050988

§ 371 Date: Mar. 18, 2025

Related U.S. Application Data
(60) Provisional application No. 63/515,583, filed on Jul. 25, 2023.

(51) Int. Cl. G01N 27/414; B33Y 80/00; G01N 27/327; G01N 33/53; G01N 33/543

(52) U.S. Cl. CPC G01N 27/4145; B33Y 80/00; G01N 27/3276; G01N 33/5308; G01N 33/5438

(57) ABSTRACT

An organic electrolyte-gated field effect transistor biosensor contains a microfluidic channel structure formed from a dielectric thermoplastic material. A biorecognition entity immobilized within the microfluidic channel allows the biosensor to detect the presence or concentration of an analyte in an electrolyte fluid placed within the channel. The dielectric material separates the microfluidic channel from the gate electrode and from the semiconductor material connecting the drain and source electrodes, and protects the gate electrode and the semiconductor material from direct contact with the electrolyte fluid in the microfluidic channel, to provide the biosensor with a high capacitance. Methods of fabricating the biosensor by monolithic 3D printing are also provided.

### FIELD

The present application is directed to biosensors. More specifically, the present application is directed to improved organic electrolyte-gated field effect transistor-based biosensors, and related methods and uses to measure the presence and quantity of analytes in biological and other samples.

### BACKGROUND

According to definitions originating from the International Union of Pure and Applied Chemistry (IUPAC), a biosensor can be considered to be a specific type of chemical sensor. Chemical sensors are considered to include two basic functional units: a receptor and a transducer. Upon interaction of an analyte with the receptor, the transducer transforms chemical information about the analyte, such as its presence or concentration, into an analytically useful signal. In biosensors, the interaction of the receptor with the analyte occurs by a biochemical mechanism, such as binding to or reaction with a protein, polynucleotide or other biological molecule (for example, an antibody, antigen, enzyme, receptor, aptamer, etc.) or by interaction of the analyte with biological material such as a cell or tissue.

One type of transducer which can be used in a chemical sensor or biosensor is a field effect transistor (FET). FETs are semiconductor-based devices and traditionally comprise an inorganic semiconductor material such as silicon which is often doped with small amounts of elements such as boron, aluminum, gallium, indium, phosphorus, arsenic, or antimony, although organic semiconductor materials can also be used. Commonly used organic semiconductor materials are characterized by an aromatic, conjugated, or otherwise delocalized π-electron system and include compounds such as rubrene, pentacene and polymers of thiophene, which may be further chemically substituted to adjust their conducting properties. Semiconductor materials can be classified as n-type, in which electrons are the major charge carriers, or p-type, in which positive holes are the major charge carriers.

A FET has three terminals or electrodes indicated as the gate, the source and the drain, respectively. A conductive channel in the semiconductor material connecting the source and the drain can allow a current to flow between the source and drain terminals. However, by applying a voltage to the gate, the conductivity of the channel, and thus the output current of the FET, can be modified. If the gate voltage undergoes changes related to interaction of an analyte with a receptor of a chemical sensor or biosensor, the resulting changes in the output current of the FET can provide an analytically useful signal reflective of information related to the analyte.

For example, in insulated-gate FETs such as MOSFETs (Metal Oxide Semiconductor FETs), the presence of an insulating dielectric layer between the gate and the conductive channel allows the FET to behave as a capacitor. Depending upon the voltage applied to the gate, an electric field can be generated which, depending on the specific composition of the FET, can increase or decrease the number of charge carriers within the conductive channel, thereby increasing or decreasing the output current flowing between the source and drain terminals. Thus, for example, if the gate is positively polarized with respect to the conductive channel, the semiconductor material of the conductive channel will be enriched with electrons such that the gate electrode takes on a net positive charge, while the semiconductor takes on a net negative charge. If the conductive channel contains a n-type semiconductor, there will be an increased number of electrons available to carry charge. Similarly, if the gate is negatively polarized with respect to the conductive channel, the semiconductor material of the conductive channel will be depleted of electrons such that the gate electrode takes on a net negative charge, while the semiconductor takes on a net positive charge. If the conductive channel contains a p-type semiconductor, the electron depletion will result in an increase in the number of positive holes available to carry charge. In each of these cases, increasing the magnitude of the gate voltage will result in an increased number of charge carriers in the conducting channel, and therefore will increase the output current flowing between the source and the drain terminals. Conversely, decreasing the magnitude of the gate voltage will result in a decreased number of charge carriers in the conducting channel, and therefore will decrease the output current flowing between the source and the drain terminals.

Electrolyte-gated FETs, or EGFETs, in their basic form contain an electrolyte as part of the dielectric layer between the gate electrode and the semiconductor material. The electrolyte may be, for example, an aqueous ion-containing solution, an ionic liquid, an ionic gel or a conducting polymer. When a biasing voltage is applied to the gate, ions in the electrolyte move towards the gate or away from the gate, depending on their charge. Thus, for example, if the gate is negatively polarized with respect to the semiconductor material as described above, positively charged cations from the electrolyte are attracted to and move towards the negatively charged gate, resulting in the formation of an electric double layer at the interface between the electrolyte and the gate. Similarly, negatively charged anions in the electrolyte are repelled by the negatively charged gate and move towards the positively charged semiconductor, resulting in the formation of a second electric double layer at the interface between the electrolyte and the semiconductor surface. Alternatively, if the gate is positively polarized with respect to the semiconductor material as described above, negatively charged anions from the electrolyte move towards the positively charged gate and positively charged cations in the electrolyte move towards the negatively charged semiconductor, again resulting in the formation of electric double layers at the respective interfaces.

The presence of the electric double layers allows formation of a high degree of charge separation between the gate and the conductive channel for a given applied gate voltage, thus providing EGFETs with a high gate capacitance. It is notable that the capacitance of the EGFET is proportionally related to ionic concentration. Thus, a change in the ionic concentration of the gating electrolyte can result in a corresponding change in the gate capacitance in the resulting electric field at a given gate voltage and in the conductivity of the conductive channel, and therefore such a change in ionic concentration can be reflected in a measurable change in the output current of the EGFET.

EGFETs containing organic semiconductor materials (EGOFETs) are well suited for use as transducers in biosensors, at least in part because they are readily incorporated into small and possibly flexible devices which can be efficiently manufactured using 3D printing technology. As a result of the high gate capacitance, EGOFETs can operate at lower applied gate voltages, making them less likely to cause electrolysis of aqueous media and therefore more compatible with biological systems. In addition, smaller changes in gate voltage or capacitance can result in larger changes in the conductivity of the conductive channel and output current, resulting in increased sensitivity. Furthermore, it is possible for an EGOFET to utilize a biological fluid like blood, plasma, urine, sweat, exhaled breath condensate or saliva as a gating electrolyte and to include at the interface between the electrolyte and either of the gate or the semiconductor a layer bearing functionality to which an analyte can selectively bind. It has been found that binding of the analyte, if present in the biological fluid, to the functionalized layer can cause changes in the gate capacitance with resulting changes in the output current which are reflective of the concentration of the analyte in the fluid. As a result, EGOFETs can be particularly useful in biosensors designed to detect the presence or concentration of an analyte of interest in an aqueous biological fluid.

Previous reports of attempts to design biosensors using EGOFETs indicate that, while it is possible to prepare biomolecule sensitive devices which demonstrate transduction changes reflecting changes in the concentration of an analyte, the reported devices have limited repeatability and high variability in correlating output from a complex sample to analyte concentration. One possible complicating factor is the contribution of pseudocapacitance to the total capacitance, resulting from charge transfer occurring between the electrolyte and the gate electrode due to electrosorption or intercalation processes or reversible faradaic redox reactions. Furthermore, known processes used to passivate the surface of semiconductor material can allow fluid infiltration from the electrolyte to the semiconductor surface, causing degradation of the surface passivation layer and leading to loss of sensitivity and poor device shelf life.

Organic electrolyte-gated FET (OEGFET) biosensors which overcome some of these deficiencies have been reported. For example, see Massey, R., et al, “Label-free detection of dopamine using aptamer enhanced organic-electrolyte gated FET sensor.” In 2019 IEEE International Conference on Flexible and Printable Sensors and Systems (FLEPS), pp. 1-3. IEEE, 2019; Massey, R., et al, “Aptamer-Enhanced Organic Electrolyte-Gated FET Biosensor for High-Specificity Detection of Cortisol.” IEEE Sensors Letters 4, no. 7 (2020): 1-4; Massey, R., et al, “A Comprehensive Modelling Approach for Bio-EDLC systems.” In 2020 IEEE Sensors, pp. 1-4. IEEE, 2020; R. Massey et al, “A System-On-Board Integrated Multi-Analyte PoC Biosensor for Combined Analysis of Saliva and Exhaled Breath”, 2022 IEEE International Engineering in Medicine and Biology Society Conference, pp. 1-6, 2022; R. Massey et al, “System-On-Board Integrated Flexible OEGFET Aptasensor for Saliva Testing of Cortisol”, 2022 IEEE International Conference on Flexible and Printable Sensors and Systems (FLEPS), pp. 1-4, 2022; and R. Massey et al, “A Low Temperature Processed, Soft-fluidic OEGFET Saliva Aptasensor for Cortisol”, IEEE Journal on Flexible Electronics, 1, no. 1, pp. 64-72, January 2022.

As described, for example, in R. S. Massey and R. Prakash, “A Low-Temperature-Processed, Soft-Fluidic OEGFET Saliva Aptasensor for Cortisol,” in IEEE Journal on Flexible Electronics, vol. 1, no. 1, pp. 64-72, January 2022, these OEGFETs incorporate a two-surface design, in which a microfluidic channel structure is sandwiched between a top layer and a bottom layer. The top layer is prepared by depositing a chromium gate electrode onto a flexible polyamide film (Kapton™) using a shadow mask, followed by a layer of poly(methyl methacrylate) (PMMA). The microfluidic channel structure is molded or stamped from polydimethylsiloxane (PDMS), cured and sealed to the PMMA of the top layer using a cross-linking process involving (3-aminopropyl)triethoxysilane (APTES). The PMMA surface of the top layer is then biofunctionalized by drop casting, with UV activation, an aptamer designed to recognize a particular analyte, such that fluid present in the microfluidic channel is exposed to the biosensing surface of the top layer. The bottom layer is prepared by depositing aluminum and chromium onto Kapton™ film using a shadow mask to form the source and drain electrodes. An organic semiconductor such as 6,13-bis(triisopropylsilylethynyl)pentacene (TIPS-pentacene) is spin deposited on the surface, annealed to connect the source and drain electrodes, and covered by multiple stacked layers of a dielectric material such as polyvinyl alcohol (PVA) and an outer layer of uncured PDMS which is contacted with the microfluidic channel and sealed by curing. Thus, both the gate electrode and the semiconductor connecting the source and drain electrodes are isolated from contact with the electrolyte fluid in the microfluidic channel by intervening dielectric material, thereby reducing or eliminating the possible impact of pseudocapacitance and avoiding damage to the semiconductor from exposure to the electrolyte fluid.

OEGFET-based biosensors manufactured in this way have been reported to show useful concentration-dependent transduction changes for analytes such as cortisol, dopamine and α-synuclein. However, these biosensors exhibit some disadvantages. For example, the fabrication process cannot be scaled up to commercial levels and introduces process variations, in part due to lack of control of the orientation of crystal growth of the organic semiconductor, resulting in varying levels of resistance to electron transport through the conductive channel and leading to inconsistency in the performance between different devices. As well, because the microfluidic channels are molded from PDMS, they can vary in thickness and fill volume from one device to another, further leading to inter-device inconsistency. The need for crosslinking of the PDMS microfluidic channel to the PMMA dielectric material also introduces the possibility of incomplete sealing of the microchannel and subsequent leakage of the electrolyte sample. In addition, the shelf life of the device is not long enough for feasible commercial use. Further, the manufacturing process is not well-suited to incorporating biorecognition molecules other than aptamers into the biosensing surface. Thus, biosensors reducing or overcoming some of these disadvantages are desirable.

### SUMMARY

In one aspect, the present application provides an organic electrolyte-gated field effect transistor biosensor containing a gate electrode, a source electrode, a drain electrode and a semiconductor material in electrical contact with the source electrode and the drain electrode. The biosensor also contains a microfluidic channel structure formed from at least one thermoplastic dielectric material and comprising at least one interior surface bearing a biorecognition entity, wherein the at least one interior surface is separated from the gate electrode, the source electrode, the drain electrode and the semiconductor material by the thermoplastic dielectric material. In at least one embodiment, the biorecognition entity is an aptamer. In at least one embodiment, the biorecognition entity is an antibody.

In at least one embodiment, the microfluidic channel structure comprises a first dielectric layer having a first surface, a second dielectric layer having a second surface parallel to and opposing the first surface and one or more sidewalls contiguous with the first dielectric layer and with the second dielectric layer so as to separate the first dielectric layer from the second dielectric layer and define a microfluidic channel between the first surface, the second surface and the one or more sidewalls. The microfluidic channel is configured to receive an electrolyte fluid such that the electrolyte fluid is in contact with the first surface and the second surface. In addition, at least one of the first surface and the second surface bears the biorecognition entity. In at least one embodiment, the first surface bears the biorecognition entity. In at least one embodiment, the second surface bears the biorecognition entity. In at least one embodiment, both the first surface and the second surface bear the biorecognition entity.

In at least one embodiment, the semiconductor material is an organic or carbon-based semiconductor material. In at least one such embodiment, the semiconductor material is selected from the group consisting of 6,13-bis(triisopropylsilylethynyl)pentacene (TIPS-pentacene), polythiophenes and copolymers thereof, metal phthalocyanines, and single-walled carbon nanotubes. In at least one such embodiment, the semiconductor material comprises 6,13-bis(triisopropylsilylethynyl)pentacene (TIPS-pentacene) or carbon nanotubes. In at least one embodiment, the semiconductor material is metal oxide semiconductor material. In at least one embodiment, the semiconductor material is formed by deposition on a self-assembled monolayer. In at least one embodiment, the self-assembled monolayer comprises octadecyltrichlorosilane.

In at least one embodiment, the thermoplastic dielectric material of the microfluidic channel structure and, in at least one embodiment, of the first and second dielectric layers and the one or more sidewalls, is one or more materials selected from the group consisting of polylactic acid (PLA), polycaprolactone (PCL), poly(ethyl methacrylate) (PEMA), polybutylene terephthalate (PBT), polyethylene terephthalate (PET), polyethylene (PE), polypropylene (PP), polystyrene (PS), polytetrafluoroethylene (PTFE), cellulose, acylated cellulose derivatives, cellulose acetate, cellulose acetate butyrate, cellulose propionate, acrylonitrile butadiene styrene (ABS), acrylonitrile styrene acrylate (ASA), polycaprolactam (PC), polybutadiene (PBD), polydimethylsiloxane (PDMS) and nylon polyamides. In at least one embodiment, the thermoplastic dielectric material comprises at least one of polylactic acid (PLA) and polycaprolactone (PCL).

In at least one embodiment, one or both of the first dielectric layer and the second dielectric layer comprise at least one of polylactic acid (PLA) and polycaprolactone (PCL). In at least one embodiment, the first dielectric layer and the second dielectric layer each comprise polylactic acid (PLA), polycaprolactone (PCL) or a mixture of PLA and PCL. In at least one embodiment, both the first dielectric layer and the second dielectric layer each comprise PCL. In at least one embodiment, both the first dielectric layer and the second dielectric layer each comprise PLA. In at least one embodiment, the first dielectric layer comprises PCL and the second dielectric layer comprises PLA. In at least one such embodiment, the first surface bears the biorecognition entity. In at least one embodiment, the first dielectric layer comprises PLA and the second dielectric layer comprises PCL. In at least one such embodiment, the second surface bears the biorecognition entity.

In at least one embodiment in which one or both of the first dielectric layer and the second dielectric layer comprise PCL, the biorecognition entity can be bound directly to the PCL at one or both of the first surface and the second surface. In at least one embodiment in which one or both of the first dielectric layer and the second dielectric layer comprise PCL, the biorecognition entity can be bound to a layer of poly(methyl methacrylate) (PMMA) at one or both of the first surface and the second surface.

In at least one embodiment in which the first dielectric layer and the second dielectric layer each comprise PLA, the first dielectric layer contains a polylactic acid—polyethylene glycol—carboxylic acid block copolymer and the first surface bears the biorecognition entity.

In at least one embodiment, the second dielectric layer contains a polylactic acid-polyethylene glycol—carboxylic acid block copolymer and the second surface bears the biorecognition entity.

In at least one embodiment, at least one of the first dielectric layer and the second dielectric layer comprises a layer comprising PLA, a layer comprising polyvinyl alcohol (PVA) and nanocrystalline cellulose, and a layer comprising PCL.

Another aspect of the present application provides a method of producing an organic electrolyte-gated field effect transistor biosensor as described herein. In at least one embodiment, the method comprises:
3D printing a thermoplastic dielectric material to form a microfluidic channel structure comprising at least one interior surface;
disposing the microfluidic channel structure between a gate electrode and a semiconductor material in electrical contact with a source electrode and a drain electrode such that the at least one interior surface is separated from the gate electrode and the semiconductor material by the dielectric material; and
binding a biorecognition entity to at least a portion of the at least one interior surface. In at least one embodiment, the microfluidic channel structure is printed three-dimensionally from the thermoplastic dielectric material as a monolithic structure.

In another aspect, the present application provides a method of analyzing an electrolyte fluid for an analyte, comprising exposing an organic electrolyte-gated field effect transistor biosensor as described herein to the electrolyte fluid and determining one or more features of the analyte. In at least one embodiment, the electrolyte fluid is a sample obtained from an environmental source. In at least one embodiment, the electrolyte fluid is a sample obtained from an agricultural source. In at least one embodiment, the electrolyte fluid is a sample obtained from a food source. In at least one embodiment, the electrolyte fluid is a sample obtained from a manufacturing process. In at least one embodiment, the electrolyte fluid is a biological fluid. In at least one embodiment, the biological fluid is one or more of blood, plasma, urine, sweat, interstitial fluid, cerebrospinal fluid, saliva or exhaled breath condensate. In at least one embodiment, the electrolyte fluid is saliva. In at least one embodiment, the electrolyte fluid is blood. In at least one embodiment, the electrolyte fluid is plasma.

In at least one embodiment, the analyte is a biomarker whose presence or concentration is indicative of a medical condition. In at least one embodiment, the analyte in the electrolyte fluid is selected from the group consisting of drugs, metabolites, hormones, neurotransmitters, enzymes, carcinogens, peptides, proteins, electrolytes, metal ions, nucleic acids and cells. In at least one embodiment, the analyte in the electrolyte fluid is an analyte useful for measuring neural function, cardiac function, liver function or kidney function or for detecting neurodegenerative conditions, cardiac conditions or disorders, blood conditions or disorders, infection, conditions related to inflammation or stress, hepatic conditions or disorders, renal conditions or disorders and cancer or precancerous conditions. In at least one embodiment, the analyte in the electrolyte fluid is selected from the group consisting of α-synuclein, β-amyloids, and tau proteins.

In at least one embodiment, exposing the biosensor to the electrolyte fluid comprises exposing the microfluidic channel of the biosensor to the electrolyte fluid. In at least one embodiment, determining one or more features of the analyte comprises determining the presence of the analyte in the electrolyte fluid or measuring the concentration of the analyte in the electrolyte fluid. In at least one embodiment, interaction of the analyte with the biorecognition entity results in one or more of formation of a charge separation region at an interface between the electrolyte fluid and at least one of the first dielectric layer and the second dielectric layer, a change in a net gate-to-channel capacitance of the biosensor, and a change in output current of the biosensor wherein the change in output current is indicative of the one or more features of the analyte in the electrolyte fluid.

### BRIEF DESCRIPTION OF THE DRAWINGS

Further features of the present invention will become apparent from the following written description and the accompanying figures, which are not necessarily drawn to scale, in which:

FIG. 1 is a top plan view of a diagrammatic representation of an embodiment of a biosensor according to the present application;

FIG. 2 is a cross-sectional view of the diagrammatic representation of FIG. 1 along line 2-2;

FIG. 3A is a cross-sectional view of a diagrammatic representation of an alternative embodiment of a biosensor according to the present application;

FIG. 3B is a cross-sectional view of a diagrammatic representation of an alternative embodiment of a biosensor according to the present application;

FIG. 4A is a cross-sectional view of a diagrammatic representation of an alternative embodiment of a biosensor according to the present application;

FIG. 4B is a cross-sectional view of a diagrammatic representation of an alternative embodiment of a biosensor according to the present application;

FIG. 5 is a cross-sectional view of a diagrammatic representation of an alternative embodiment of a biosensor according to the present application;

FIG. 6A is a front perspective view of a diagrammatic representation of an alternative embodiment of a biosensor according to the present application;

FIG. 6B is a cross-sectional view of the diagrammatic representation of FIG. 6A along line B-B;

FIG. 6C is a cross-sectional view of the diagrammatic representation of FIG. 6A along line C-C;

FIG. 7A is a cross-sectional view of a diagrammatic representation of an alternative embodiment of a biosensor according to the present application;

FIG. 7B is a cross-sectional view of a diagrammatic representation of an alternative embodiment of a biosensor according to the present application;

FIG. 8A is a top plan view of a diagrammatic representation of an alternative embodiment of a microfluidic channel of a biosensor according to the present application;

FIG. 8B is a top plan view of a diagrammatic representation of an alternative embodiment of a microfluidic channel of a biosensor according to the present application;

FIG. 8C is a top plan view of a diagrammatic representation of an alternative embodiment of a microfluidic channel of a biosensor according to the present application;

FIG. 8D is a top plan view of a diagrammatic representation of an alternative embodiment of a microfluidic channel of a biosensor according to the present application;

FIG. 9A is a diagrammatic representation of a binding mode by which a biorecognition entity of an embodiment of a biosensor according to the present application can bind to an analyte;

FIG. 9B is a diagrammatic representation of an alternative binding mode by which a biorecognition entity of an embodiment of a biosensor according to the present application can bind to an analyte;

FIG. 9C is a diagrammatic representation of another alternative binding mode by which a biorecognition entity of an embodiment of a biosensor according to the present application can bind to an analyte;

FIG. 10A is a graph showing the variation of device output current (I_{DS}) with input channel voltage (V_{DS}) at various applied gate voltages (VGS) for an embodiment of the present biosensor bearing an aptamer as a biorecognition entity in the presence of deionized water as an electrolyte;

FIG. 10B is a graph showing the variation of device output current (I_{DS}) with applied gate bias (V_{GS}) in the absence of electrolyte at various drain-source voltages (VDS), to measure the device transfer function for the embodiment of FIG. 10A;

FIG. 10C is a graph showing the variation of device output current with input channel voltage (V_{SD}) at various applied gate voltages (VSG) for an embodiment of the present biosensor including polycaprolactone (PCL) as the dielectric material and bearing an antibody as a biorecognition entity in the presence of deionized water as an electrolyte;

FIG. 10D is a plot showing the variation of channel current and transconductance (transfer function) and capacitance with gate modulation voltage (VSG) for the embodiment of FIG. 10C;

FIG. 10E is a graph showing the variation of device output current (I_{SD}) with input channel voltage (V_{SD}) at various applied gate voltages (VSG) for an embodiment of the present biosensor including polylactic acid (PLA) as the dielectric material and bearing an antibody as a biorecognition entity in the presence of deionized water as an electrolyte;

FIG. 10F is a plot showing the variation of channel current and transconductance (transfer function) and capacitance with gate modulation voltage (VSG) for the embodiment of FIG. 10E;

FIG. 11 is a graph showing the variation of device current with channel voltage (V_{DS}) of various embodiments of the present biosensor in which the semiconductor channel has been prepared by various methods;

FIG. 12 is a graph showing the variation of device current with channel voltage for an embodiment of the present biosensor bearing an aptamer recognizing cortisol as a biorecognition entity when exposed to synthetic buffer containing varying concentrations of cortisol;

FIG. 13 is a graph showing the variation of device current with channel voltage for an embodiment of the present biosensor bearing an aptamer recognizing α-synuclein (α-Syn) as a biorecognition entity when exposed to synthetic buffer containing varying concentrations of α-synuclein;

FIG. 14 is a plot showing device current and capacitance for an embodiment of the present biosensor bearing an aptamer recognizing α-synuclein (α-Syn) as a biorecognition entity when exposed to saliva containing varying concentrations of α-synuclein;

FIG. 15A is a graph showing the variation of device output current (I_{SD}) with input channel voltage (V_{SD}) at various applied gate voltages (VSG) for an embodiment of the present biosensor including single walled carbon nanotubes as the semiconductor and polycaprolactone (PCL) as the dielectric material in the presence of deionized water as the electrolyte;

FIG. 15B is a graph showing the variation of device output current (I_{SD}) with applied gate voltage (V_{SG}) at various drain-source voltages (VSD) to measure the device transfer function of the embodiment of FIG. 15A in the presence of deionized water as the electrolyte;

FIG. 15C is a graph showing the variation of device output current (I_{SD}) with input channel voltage (V_{SD}) at various applied gate voltages (VSG) for the embodiment of FIG. 15A in the presence of Tris-acetate-EDTA (TAE) buffer as the electrolyte;

FIG. 15D is a graph showing the variation of device output current (I_{SD}) with applied gate voltage (V_{SG}) at various drain-source voltages (VSD) to measure the device transfer function of the embodiment of FIG. 15A in the presence of TAE buffer as the electrolyte;

FIG. 16A is a plot showing device current and capacitance for an embodiment of the present biosensor including polycaprolactone (PCL) as the dielectric material and bearing an aptamer recognizing α-synuclein as a biorecognition entity when exposed to TAE buffer solutions containing varying concentrations of α-synuclein;

FIG. 16B is a plot showing device current and capacitance for an alternative embodiment of the present biosensor including polycaprolactone (PCL) as the dielectric material and bearing a monoclonal antibody recognizing amyloid β-42 as a biorecognition entity when exposed to TAE buffer solutions containing varying concentrations of amyloid β-42;

FIG. 16C is a plot showing device current and capacitance for the embodiment of FIG. 16A when exposed to real saliva supernatant (RSN) samples containing varying concentrations of α-synuclein;

FIG. 16D is a plot showing device current and capacitance for the embodiment of FIG. 16B when exposed to RSN samples containing varying concentrations of amyloid β-42;

FIG. 16E is a plot showing device current and capacitance for the embodiment of FIG. 16A when exposed to real blood serum (indicated as “WTS”) samples obtained from mice and containing varying concentrations of α-synuclein; and

FIG. 16F is a plot showing device current and capacitance for the embodiment of FIG. 16B when exposed to WTS serum samples containing varying concentrations of amyloid β-42.

### DETAILED DESCRIPTION

In one aspect, the present application provides an organic electrolyte-gated field effect transistor biosensor containing a gate electrode, a source electrode, a drain electrode, and a semiconductor material in electrical contact with the source electrode and the drain electrode. The biosensor also comprises a microfluidic channel structure formed from a dielectric material and comprising at least one interior surface bearing a biorecognition entity.

In at least one embodiment, the microfluidic channel structure contains a first dielectric layer having a first surface, a second dielectric layer having a second surface which is parallel to and opposite the first surface of the first dielectric layer, and one or more sidewalls contiguous with the first and second dielectric layers and separating the first surface from the second surface to define a microfluidic channel therebetween. In at least one embodiment, the first dielectric layer separates the gate electrode from the microfluidic channel. In at least one embodiment, the second dielectric layer separates the semiconductor material from the microfluidic channel. In at least one embodiment, the first dielectric layer separates the gate electrode from the microfluidic channel and the second dielectric layer separates the semiconductor material from the microfluidic channel, such that the gate electrode is separated from the semiconductor material by the first dielectric layer, the microfluidic channel and the second dielectric layer. This arrangement can provide a high capacitance between the gate electrode and the semiconductor material of the organic electrolyte-gated field effect transistor biosensor.

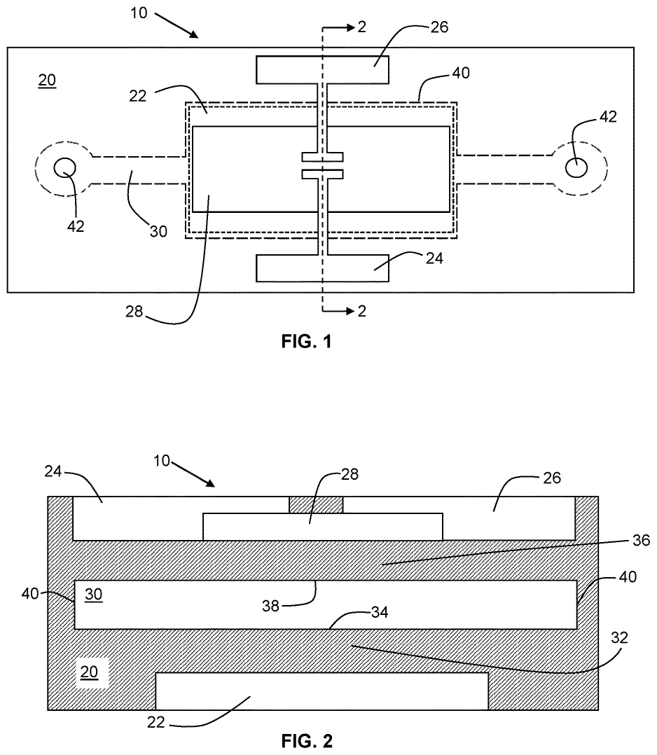

FIGS. 1 and 2 show a diagrammatic representation of such an embodiment of the present biosensor. Biosensor 10 includes gate electrode 22, source electrode 24 and drain electrode 26 connected by semiconductor material 28, and a microfluidic channel structure formed from dielectric material 20. The microfluidic channel structure includes microfluidic channel 30 defined by first dielectric layer 32 between the gate 22 and the microfluidic channel 30, with first surface 34 in contact with the microfluidic channel 30, second dielectric layer 36 between the semiconductor material 28 and the microfluidic channel 30, with second surface 38 in contact with the microfluidic channel 30, and one or more sidewalls 40 separating the first surface 34 from the second surface 38. As will be understood by the skilled person, the one or more sidewalls 40 can form a contiguous sidewall defining the perimeter of microfluidic channel 30. The microfluidic channel structure also includes two or more openings indicated generally at 42 through which an electrolyte fluid can be injected into or removed from microfluidic channel 30, or which provide an entrance and an exit through which an electrolyte fluid can flow through the microfluidic channel 30.

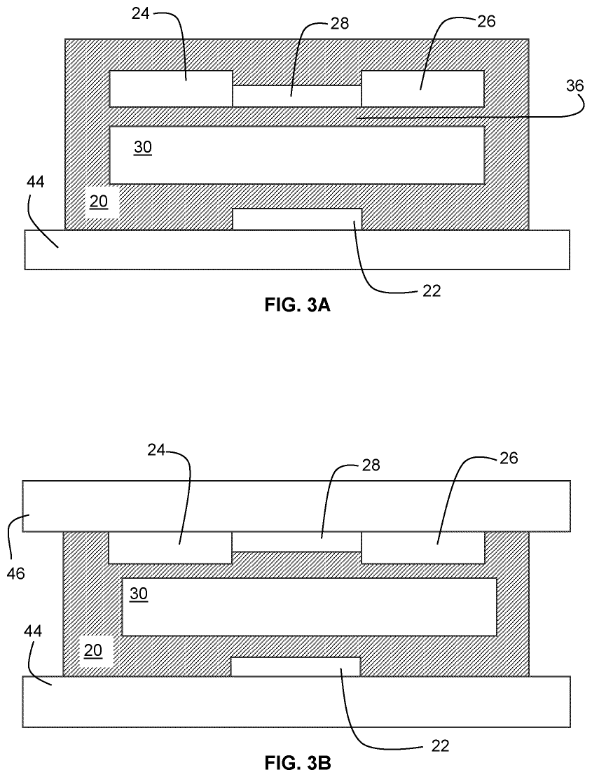

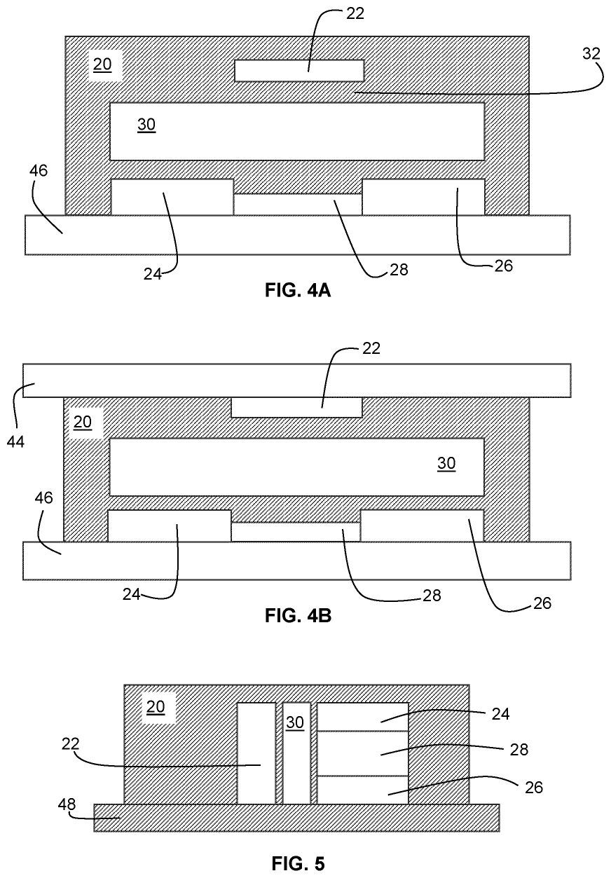

In at least one embodiment, gate electrode 22 is situated at the bottom of the biosensor as the biosensor would be positioned during use, as shown in FIGS. 1 and 2. FIG. 3A diagrammatically illustrates an alternative embodiment including gate substrate 44 supporting gate electrode 22. Another alternative embodiment, diagrammatically illustrated in FIG. 3B, further includes source/drain substrate 46 supporting source electrode 24, drain electrode 26 and semiconductor material 28. In further alternative embodiments, diagrammatically illustrated in FIGS. 4A and 4B, the gate electrode is situated at the top of the biosensor as the biosensor would be positioned during use. FIG. 5 diagrammatically illustrates an embodiment in which the gate electrode 22, microfluidic channel 30, source electrode 24, drain electrode 26 and semiconductor material 28 are supported by a substrate 48 prepared from dielectric material 20 and oriented perpendicularly to the orientation shown in FIGS. 1 to 4B.

In such embodiments, the distance between the gate electrode 22 and the semiconductor material 28 through the microfluidic channel 30 can be relatively small compared to the area of the gate electrode, the semiconductor material and the microfluidic channel. Without being bound by theory, it is contemplated that such embodiments can provide a small capacitive distance between the gate electrode and the semiconductor material and a relatively large conductive channel between the source and drain electrodes while providing a relatively small diffusion distance between the first and second surfaces. In at least one embodiment, the area of the biosensor device can range from about 500 μm² to about 1000 μm². In at least one embodiment, the length of the conductive channel formed between the source and drain electrodes through the semiconductor material can range from about 20 μm to about 200 μm.

In at least one embodiment, the thickness of the first dielectric layer, between the gate electrode and the first surface, ranges from about 50 nm to about 500 nm. In at least one embodiment, the thickness of the first dielectric layer, between the gate electrode and the first surface, is about 200 nm. In at least one embodiment, the thickness of the second dielectric layer, between the semiconductor and the second surface, ranges from about 50 nm to about 500 nm. In at least one embodiment, the thickness of the second dielectric layer, between the semiconductor and the second surface, is about 200 nm.

In at least one embodiment, the distance between the first surface 34 and the second surface 38 within the microfluidic channel 30 can range from about 100 μm to about 1000 μm. In at least one embodiment, the distance between the first surface 34 and the second surface 38 within the microfluidic channel 30 can range from about 100 μm to about 600 μm. In at least one embodiment, the distance between the first surface 34 and the second surface 38 within the microfluidic channel 30 can range from about 300 μm to about 600 μm. In at least one embodiment, the distance between the first surface 34 and the second surface 38 within the microfluidic channel 30 can range from about 400 μm to about 600 μm. In at least one embodiment, the distance between the first surface 34 and the second surface 38 within the microfluidic channel 30 is about 300 μm. In at least one embodiment, the distance between the first surface 34 and the second surface 38 within the microfluidic channel 30 is about 400 μm. In at least one embodiment, the distance between the first surface 34 and the second surface 38 within the microfluidic channel 30 is about 500 μm. In at least one embodiment, the distance between the first surface 34 and the second surface 38 within the microfluidic channel 30 is about 550 μm. In at least one embodiment, the distance between the first surface 34 and the second surface 38 within the microfluidic channel 30 is about 600 μm.

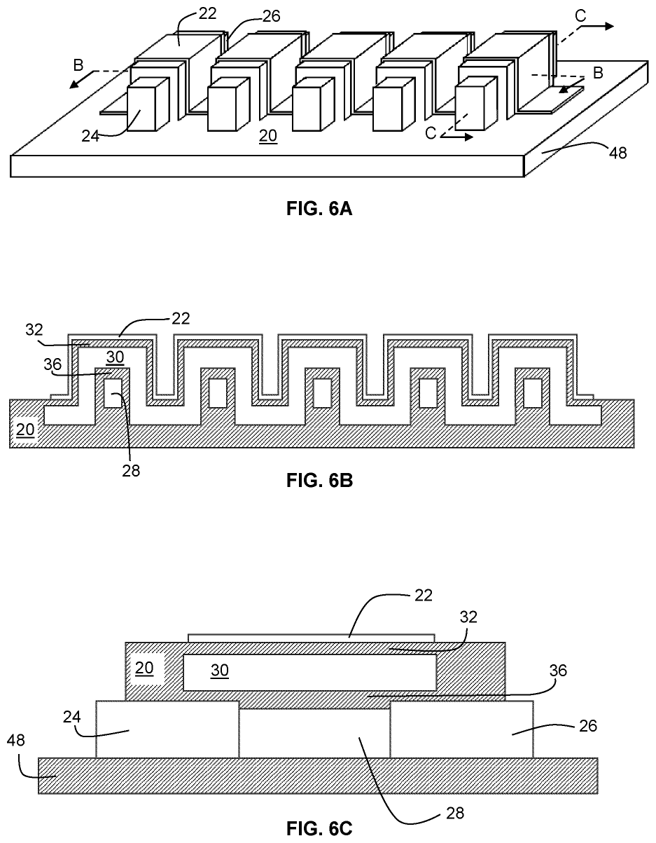

FIGS. 6A to 6C diagrammatically illustrate an embodiment of the present biosensor in which several source electrodes 24 and drain electrodes 26 are deposited on a substrate 48 prepared from dielectric material 20. A microfluidic channel structure is formed from the dielectric material 20 to include a meandering microfluidic channel 30, second dielectric layer 36, which separates semiconductor material 28 from microfluidic channel 30, and first dielectric layer 32, which separates microfluidic channel 30 from gate electrode 22, deposited on the outer surface of first dielectric layer 32. It will be clear to the person skilled in the art that the individual source electrodes can be electrically connected to each other such that they act together as a single source electrode. Similarly, the individual drain electrodes can be electrically connected to each other such that they act together as a single drain electrode.

In at least one embodiment of the present biosensor, the microfluidic channel can be positioned adjacent to the semiconductor material or adjacent to the gate electrode. In such embodiments, the semiconductor material and the gate electrode are separated from each other by a single dielectric layer, and a change in the capacitance of the microfluidic channel can have a fringe or peripheral capacitance effect on the charge distribution between the gate electrode and the semiconductor material 28 which can be reflected in in the output current of the field effect transistor, as will be understood by one skilled in the art.

In at least one embodiment of the present biosensor, the electrolyte gating function can be physically separated from the transistor function. Thus, in at least one such embodiment, the first dielectric layer of the microfluidic channel structure separates a first plate of a capacitor from the microfluidic channel and the second dielectric layer separates a second plate of the capacitor from the microfluidic channel, such that the first and second plates of the capacitor are separated from each other by the first dielectric layer, the microfluidic channel and the second dielectric layer. In such embodiments, one of the first plate or the second plate of the capacitor can be electrically connected to the gate electrode of a field effect transistor containing a gate electrode, a source electrode, a drain electrode, a semiconductor material connecting the source electrode and the drain electrode, and a dielectric layer between the gate electrode and the semiconductor material, as is well known in the art. In this way, a change in the voltage of the capacitor related to a change in capacitance of the microfluidic channel can be reflected in the voltage of the gate electrode of the field effect transistor and thus in its output current.

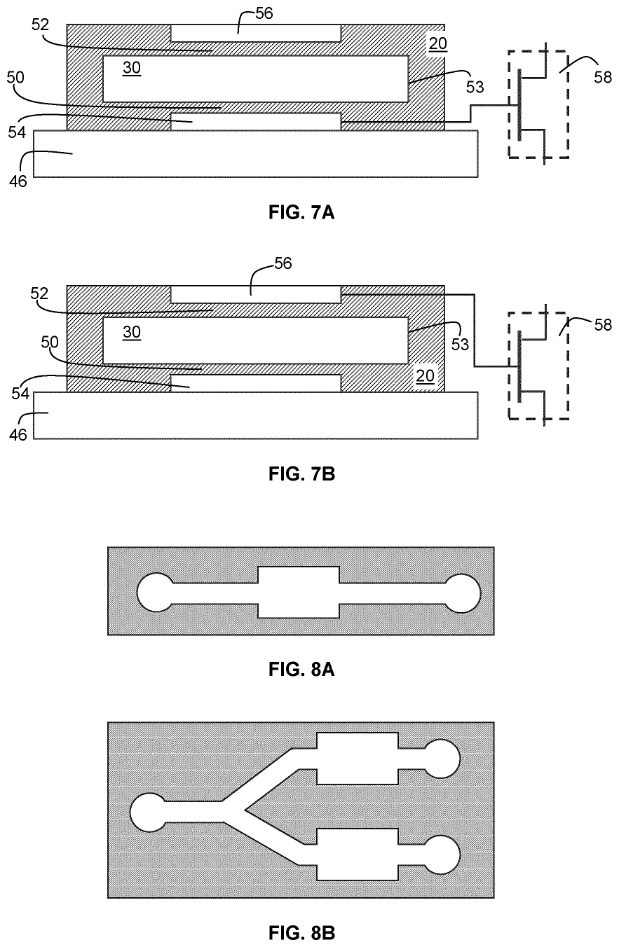

Such an embodiment is diagrammatically illustrated in FIGS. 7A and 7B. The illustrated embodiment contains a microfluidic channel structure formed from a dielectric material 20 and comprising first dielectric layer 50, second dielectric layer 52, and one or more sidewalls 53, which together define microfluidic channel 30. The microfluidic channel structure is positioned between a first capacitor plate 54 and a second capacitor plate 56, such that the microfluidic channel 30 is separated from the first capacitor plate 54 by the first dielectric layer 50 and from the second capacitor plate 56 by the second dielectric layer 52.

Either the first capacitor plate 54 (FIG. 7A) or the second capacitor plate 56 (FIG. 7B) can be electrically connected to the gate electrode of a field effect transistor, generally indicated at 58, such that changes in capacitance and/or voltage of the capacitor can affect the gate voltage and thus the output current of the field effect transistor.

As is well understood in the art, the gate, source and drain electrodes are made of any suitable electrically conductive material. In at least one embodiment, the electrically conductive material is a metal, including, but not limited to, aluminum, chromium, gold, platinum and other metals known in the art. In at least one embodiment, the electrically conductive material is a non-metallic conductive element, including, but not limited to, graphite, graphene, carbon nanotubes, and other conductive allotropic forms of carbon. In at least one embodiment, the electrically conductive material is a conductive compound, including, but not limited to, a conductive polymer. Suitable conductive polymers are well known in the art and include, but are not limited to, polyacetylenes, polyphenylene vinylenes, polypyrroles, polythiophenes, polyanilines, polyphenylene sulfides, copolymers thereof, and other suitable polymers well known to those skilled in the art. In at least one embodiment, the conductive polymer is poly(3,4-ethylenedioxythiophene) polystyrene sulfonate (PEDOT:PSS).

In at least one embodiment, the gate, source and drain electrodes are deposited on a substrate by any suitable technique known in the art, including, but not limited to, spin coating, deposition from solution and printing, including, but not limited to, screen printing, ink-jet printing, aerosol jet printing, transfer foil printing, and printing through a shadow mask.

In at least one embodiment, the substrate is a flexible imide film, such as Kapton™ film. In at least one embodiment, the substrate is an outer upper surface of either the first dielectric layer or the second dielectric layer of the microfluidic channel structure. For example, in the embodiment shown in FIG. 3A, the source electrode 24, the drain electrode 26 and the semiconductor material 28 can be deposited on an outer surface of second dielectric layer 36. Similarly, in the arrangement shown in FIG. 4A, the gate electrode 22 can be deposited on an outer surface of first dielectric layer 32.

In at least one embodiment, the thickness of the gate, source and drain electrodes deposited on the substrate can range from about 100 nm to about 10 μm. In at least one embodiment, the thickness of the gate, source and drain electrodes deposited on the substrate can range from about 100 nm to about 5 μm. In at least one embodiment in which the gate, source and drain electrodes comprise a metal, the thickness of the electrodes deposited on the substrate can range from about 100 nm to about 500 nm. In at least one embodiment in which the gate, source and drain electrodes comprise a conductive polymer, the thickness of the electrodes deposited on the substrate can range from about 200 nm to about 5 μm. The skilled person would be able to select suitable shapes, sizes and relative positions of electrodes for particular embodiments, and be able to design and prepare such electrodes in view of the teaching herein.

In at least one embodiment, the semiconductor material comprises an inorganic semiconductor. In at least one embodiment, the inorganic semiconductor is silicon doped with elements such as boron, aluminum, gallium, indium, phosphorus, arsenic or antimony.

In at least one embodiment, the inorganic semiconductor is a semiconductive metal oxide including, but not limited to, indium gallium zinc oxide (IGZO), antimony tin oxide (ATO), nickel oxides (e.g., NiOₓ), titanium oxides (e.g., TiOₓ), indium oxides (e.g., InOₓ), zinc oxides (e.g., ZnOₓ), tin oxides (e.g., SnOₓ), copper oxides (e.g., CuOₓ), copper aluminum oxides (e.g., CuAlOₓ), chromium oxides (e.g., CrOₓ), cobalt oxides (e.g., CoOₓ), tungsten oxides (e.g., WOₓ), cerium oxides (e.g., CeOₓ), niobium oxides (e.g., NbOₓ), molybdenum oxides (e.g., MoOₓ), silver oxides (e.g., AgOₓ), gallium oxides (e.g., GaOₓ), and other semiconductive metal oxides well known in the art.

In at least one embodiment, the semiconductor material comprises any suitable organic or carbon-based semiconductor material known in the art, including, but not limited to, polycyclic aromatic hydrocarbons such as rubrene, anthracene and pentacene, metal phthalocyanines, carbon nanotubes, conjugated polymers such as polythiophenes, polyphenylenevinylene and copolymers of carbazole-dithiophene-benzothiadiazole, and block co-polymers of such conjugated polymers. In at least one embodiment, the organic or carbon-based semiconductors include, but are not limited to, 6,13-bis(triisopropylsilyl-ethynyl)pentacene (TIPS-pentacene), polythiophenes and copolymers thereof including, but not limited to, poly(3-hexylthiophene) (P3HT), 1,4-diketopyrrolo[3,4-c]pyrrole-thieno[3,2-b]thiophene (DPP-TTT) and dinaphthothienothiophene (DNTT), metal phthalocyanines including, but not limited to, copper phthalocyanine (CuPC), and single-walled carbon nanotubes.

In at least one embodiment, the semiconductor material is deposited on a highly ordered material layer. Without being bound by theory, it is contemplated that when the semiconductor material is an organic or carbon-based semiconductor material, the directionality of the organic semiconductor crystals formed or of the carbon nanotubes can be better controlled, resulting in better consistency between devices and improved charge mobility through the organic or carbon-based semiconductor material. In at least one such embodiment, the highly ordered material layer is a self-assembled monolayer. In at least one embodiment, the monolayer is a layer of octadecyltrichlorosilane which is one molecule in thickness.

The microfluidic channel structure, including the first and second dielectric layers, comprises a dielectric material. In at least one embodiment, the dielectric material is a heat meltable thermoplastic material which can be conveniently shaped by 3D printing or extrusion and can be adhered to a separate structure prepared from the thermoplastic material. In at least one embodiment, the thermoplastic material surfaces to be adhered to each other can be adhered by methods including, but not limited to, partial melting of the material of the adhering surfaces, crosslinking or the use of solvents to partially dissolve material of the adhering surfaces. In at least one embodiment, the dielectric material can be annealed to reduce structural defects and improve dimensional stability, resulting in better inter-device consistency during manufacturing. Suitable dielectric materials include, but are not limited to, thermoplastic polymers including, but not limited to, polydienes, polycarbonates, polyimides, polyamides, polyacrylates, polyethers, polyurethanes, polyketones, polyhalodienes, polysiloxanes, polyolefins, fluoropolymers, polyesters, polysaccharides, and polyvinyls, and copolymers thereof. In at least one embodiment, the dielectric material includes but is not limited to polylactic acid (PLA), polycaprolactone (PCL), poly(ethyl methacrylate) (PEMA), polybutylene terephthalate (PBT), polyethylene terephthalate (PET), polyethylene (PE), polypropylene (PP), polystyrene (PS), polytetrafluoroethylene (PTFE, Teflon™), cellulose and acylated cellulose derivatives such as cellulose acetate, cellulose acetate butyrate and cellulose propionate, acrylonitrile butadiene styrene (ABS), acrylonitrile styrene acrylate (ASA), polycaprolactam (PC), polybutadiene (PBD), polydimethylsiloxane (PDMS), nylon polyamides, and other polymers well known in the art. In at least one embodiment, the dielectric material includes but is not limited to polylactic acid (PLA), polycaprolactone (PCL), poly(ethyl methacrylate) (PEMA), polybutylene terephthalate (PBT), polyethylene terephthalate (PET), polyethylene (PE), polypropylene (PP), polystyrene (PS), polytetrafluoroethylene (PTFE), cellulose, cellulose acetate, cellulose acetate butyrate, cellulose propionate, acrylonitrile butadiene styrene (ABS), acrylonitrile styrene acrylate (ASA), polycaprolactam (PC), polybutadiene (PBD) and nylon polyamides.

In at least one embodiment, the dielectric material is polylactic acid (PLA). Without being bound by theory, it is contemplated that PLA is biocompatible, is hydrophobic and resistant to swelling in contact with aqueous fluids, and can be functionalized with antibodies, as described in further detail below. In addition, PLA can be 3D printed and annealed, and can interact well with metals and other polymeric materials, reducing the complexity of the fabrication process. Furthermore, PLA has dielectric and resistivity properties suitable for use in an electrolyte gated field effect transistor.

In at least one embodiment, the dielectric material is polycaprolactone (PCL). Without being bound by theory, it is contemplated that PCL is biodegradable, is hydrophobic and resistant to swelling in contact with aqueous fluids, can be readily processed at a relatively low temperature, has dielectric and resistivity properties suitable for use in an electrolyte gated field effect transistor, and can be readily covalently bonded to a biorecognition entity containing a free amino group, such as an aptamer or an antibody, as further described below.

In at least one embodiment, each of the first dielectric layer and the second dielectric layer comprises polylactic acid (PLA), polycaprolactone (PCL) or a mixture of PLA and PCL. In at least one embodiment, each of the first dielectric layer and the second dielectric layer comprises PCL. In at least one embodiment, each of the first dielectric layer and the second dielectric layer comprises PLA. In at least one embodiment, the first dielectric layer comprises PCL and the second dielectric layer comprises PLA. In at least one embodiment, the first dielectric layer comprises PLA and the second dielectric layer comprises PCL.

In at least one embodiment, the first dielectric layer and/or the second dielectric layer can include a plurality of layers, each comprising a different material. In at least one embodiment, the second dielectric layer can include a layer of PLA and a layer of polyvinyl alcohol (PVA) on which a monolayer of octadecyltrichlorosilane (OTS) is deposited. As discussed above, the presence of the OTS monolayer can provide a base on which an organic or carbon-based semiconductor material can be deposited with improved control of the directionality of the organic semiconductor crystals or carbon nanotubes, resulting in improved consistency between devices.

In at least one embodiment, at least one of the first dielectric layer and the second dielectric layer can include a trilayer dielectric material comprising PLA, polyvinyl alcohol (PVA) containing cellulose nanocrystals, and thermally crosslinked PCL. In at least one embodiment, the second dielectric layer can include the trilayer dielectric material comprising PLA, polyvinyl alcohol (PVA) containing cellulose nanocrystals, and thermally crosslinked PCL. In at least one such embodiment, the trilayer material comprises a layer of PLA, on which the semiconductor material can be deposited as described herein, a layer of PVA containing cellulose nanocrystals deposited on the PLA layer opposite to the side of the PLA layer on which the semiconductor material can be deposited, and a layer of PCL on the PVA-cellulose layer opposite to the side of the PVA-cellulose layer which is bonded to the PLA layer, such that the PCL layer forms the second surface of the second dielectric layer and can be bonded to a biorecognition entity as described herein. Without being bound by theory, it is contemplated that embodiments of the present device containing such a trilayer dielectric layer can have a longer shelf life than embodiments lacking the trilayer dielectric layer.

In at least one embodiment, at least one of the first dielectric layer and the second dielectric layer comprises a coating layer on at least one of the first and second surfaces. In at least one such embodiment, the coating layer is prepared from a material which facilitates binding of at least one of the first surface and the second surface to a biorecognition entity as discussed below. Alternatively, at least one of the first dielectric layer and the second dielectric layer may be prepared from such a material, so as to facilitate binding of the respective first or second surface to the biorecognition entity. Such materials are well known in the art, and include, but are not limited to, polydimethylsiloxane (PDMS), poly(methyl methacrylate) (PMMA), and fluoropolymers including, but not limited to, CYTOP™, Teflon™ and polyvinylidene fluoride (PVDF).

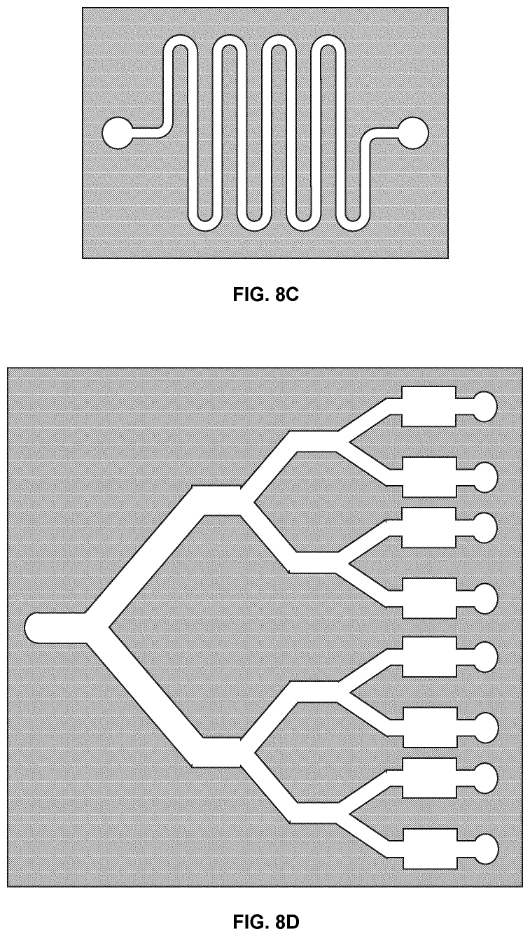

The microfluidic channel is configured to receive an electrolyte fluid such that the electrolyte fluid is in contact with the interior surface, including the first and second surfaces. In at least one embodiment, the electrolyte fluid is a sample obtained from an environmental source. In at least one embodiment, the electrolyte fluid is a sample obtained from an agricultural source. In at least one embodiment, the electrolyte fluid is a sample obtained from a food source. In at least one embodiment, the electrolyte fluid is a sample obtained from a manufacturing process. In at least one embodiment, the electrolyte fluid is a biological sample or fluid, including, but not limited to, blood, plasma, urine, sweat, interstitial fluid, cerebrospinal fluid, exhaled breath condensate and saliva. In at least one embodiment, the electrolyte fluid is saliva. In at least one embodiment, the electrolyte fluid is blood. In at least one embodiment, the electrolyte fluid is plasma. In at least one embodiment, the electrolyte fluid is a control fluid which may be used to calibrate the biosensor. The microfluidic channel can have any shape or configuration that would allow the channel to receive the electrolyte fluid so that the electrolyte fluid is in contact with the interior surface. Suitable configurations of microfluidic channels include, but are not limited to, those shown in FIGS. 8A to 8D. Other suitable configurations will be readily identified and prepared by one skilled in the art.

In at least one embodiment, the biosensor contains a calibration well for use in calibrating and standardizing the output current of the biosensor. An embodiment of a microfluidic channel containing a sample well and a calibration well is shown in FIG. 8B. In at least one embodiment, the calibration well contains a reference electrolyte of known concentration. In at least one embodiment, measurement of the output current when the reference electrolyte is present in the microfluidic channel allows calibration of the device and normalization of intra-device variations.

At least one of the first surface 34 and the second surface 38 in the microfluidic channel bears a biorecognition entity which recognizes and interacts with an analyte in the electrolyte fluid. Without being bound by theory, it is believed that the interaction results in the formation of a charge separation region in addition to the electrolytic double layer at the solid dielectric—electrolyte fluid interface, leading to changes in the net gate-to-channel capacitance of the device, thereby leading to measurable changes in the output current of the biosensor which can reflect the presence or the concentration of the analyte.

Suitable biorecognition entities are well known in the art and include, but are not limited to, antibodies, antigens, enzymes, receptors, aptamers, ligands, and the like. In at least one embodiment, the biorecognition entity is an aptamer. As used herein, and as known in the art, the term “aptamer” is intended to refer to a single-stranded polydeoxyribo-nucleotide or polyribonucleotide which folds into a defined three-dimensional structure, and which is capable of recognizing and binding to a specific target, including, but not limited to, a protein or a small molecule. In at least one embodiment, an aptamer can comprise nucleotide residues which are natural (i.e. which contain a ribose or deoxyribose moiety bearing one of the naturally occurring bases adenine, thymine, cytosine, guanine or uracil and bonded to an adjacent nucleotide residue through a monophosphate linkage between the 5′-hydroxyl group of one ribose or deoxyribose moiety and the 3-hydroxyl group of the adjacent ribose or deoxyribose moiety, as understood in the art). In at least one embodiment, an aptamer can comprise nucleotide residues which are non-natural, or which have been chemically modified, as understood in the art. In at least one embodiment, the biorecognition entity is an antibody.

In at least one embodiment, the analyte is a biomarker whose presence or concentration is indicative of a medical condition. Suitable analytes include, but are not limited to, drugs, metabolites, hormones, neurotransmitters, enzymes, carcinogens, peptides, proteins, electrolytes, metal ions, nucleic acids, cells, and other chemical or biochemical entities which it may be desirable to measure in an electrolyte fluid, including, but not limited to, a biological fluid. In at least one embodiment, the analyte is an analyte useful for measuring neural function or detecting neurodegenerative conditions, including, but not limited to, dopamine, α-synuclein, β-amyloids, and tau proteins. In at least one embodiment, the analyte useful for measuring neural function or detecting neurodegenerative conditions is one or more of dopamine and α-synuclein, which are useful analytes for detecting Parkinson's disease. In at least one embodiment, the analyte useful for measuring neural function or detecting neurodegenerative conditions is one or more of β-amyloids, and tau proteins, which are useful analytes for detecting Alzheimer's disease.

In at least one embodiment, the analyte is an analyte useful for measuring cardiac function or detecting cardiac conditions or disorders, including, but not limited to, B-type natriuretic peptide (BNP), N-terminal pro-B-type natriuretic peptide (NT-proBNP), troponin, and creatine kinase. In at least one embodiment, the analyte is an analyte useful for detecting blood conditions or disorders, including, but not limited to, bilirubin, and ferritin. In at least one embodiment, the analyte is an analyte useful for detecting infection or conditions or disorders related to inflammation or stress, including, but not limited to, cortisol, lactate, C-reactive protein, interleukins, tumor necrosis factor (TNF), procalcitonin, and inflammatory cytokines. In at least one embodiment, the analyte is an analyte useful for measuring liver function or detecting hepatic conditions or disorders, including, but not limited to, bilirubin, alanine aminotransferase, aspartate aminotransferase, gamma glutamyl transferase (GGT), albumin and carcinoembryonic antigen. In at least one embodiment, the analyte is an analyte useful for measuring kidney function or detecting renal conditions or disorders, including, but not limited to, uric acid, creatinine, and urea. In at least one embodiment, the analyte is an analyte useful for detecting cancer or precancerous conditions, including, but not limited to, tumor necrosis factor (TNF), prostate specific antigen (PSA), carcinoembryonic antigen, tumor associated antigens, and inflammatory cytokines.

As used herein and unless otherwise indicated, the terms “bear(s)” and “bearing”, when used in relation to a surface bearing an entity, including, but not limited to, a biorecognition entity, are intended to mean that the entity is attached to or incorporated into the surface such that the entity is exposed to fluid in contact with the surface but is not readily removed or washed from the surface by contact with such fluid. In at least one embodiment, the biorecognition entity is covalently or non-covalently bonded to the surface. In at least one embodiment, the biorecognition entity is covalently or non-covalently bonded to at least one intermediate entity which itself is covalently or non-covalently bonded to the surface. In at least one embodiment, the surface integrally comprises at least one of the at least one intermediate entity which is covalently or non-covalently bonded to the biorecognition entity. As a non-limiting example, the surface can incorporate or be bonded to a biorecognition entity which can directly recognize and bind to an analyte, or which can recognize and bind to at least one additional biorecognition entity which itself directly recognizes and binds to the analyte.

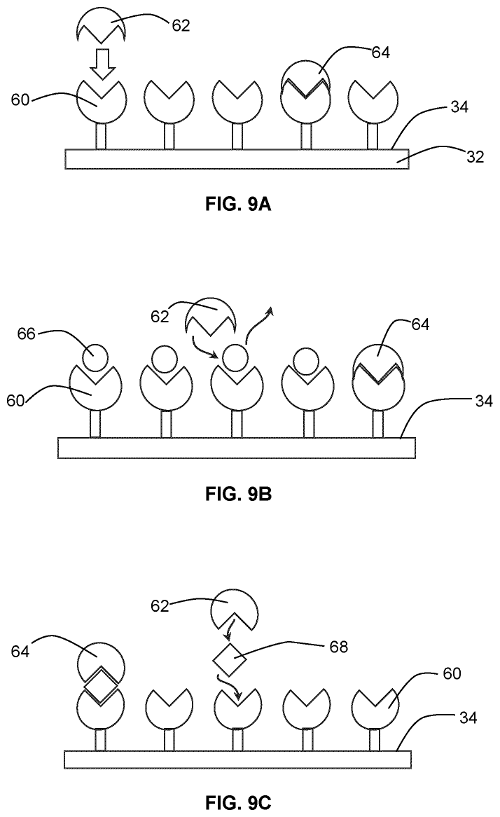

FIGS. 9A to 9C diagrammatically illustrate recognition and binding of an analyte 62 by biorecognition entities 60. While FIGS. 9A to 9C indicate for convenience that the biorecognition entities 60 are borne within microfluidic channel 30 on first surface 34 of first dielectric layer 32, it is equally contemplated that the biorecognition entities 60 can be borne within microfluidic channel 30 on second surface 38 of second dielectric layer 36, or on both first surface 34 and second surface 38.

FIG. 9A diagrammatically illustrates direct recognition and binding of free analyte 62 to biorecognition entity 60 to form bound analyte 64. FIG. 9B diagrammatically illustrates a competition binding mode, in which biorecognition entity 60 is initially bound to competing ligand 66, which is displaced from biorecognition entity 60 by analyte 62 to form bound analyte 64. FIG. 9C diagrammatically illustrates a binding mode in which both biorecognition entity 60 and analyte 62 recognize and bind a secondary entity 68, to form bound analyte 64. As will be clear to those skilled in the art, secondary entity 68 can be a ligand which is recognized and bound to the biorecognition entity 60 and which itself binds directly to analyte 62 or which facilitates binding of biorecognition entity 60 to analyte 62. Alternatively, secondary entity 68 can be a biorecognition entity which itself recognizes and binds analyte 62 and which either recognizes and binds, or is recognized and bound by, biorecognition entity 60. Other binding modes will be apparent to and readily implemented by the skilled person in view of the teaching of the present description.

The at least one of the first surface and the second surface can be configured to bear the biorecognition entity by methods well known in the art. In at least one embodiment, when the biorecognition entity is an aptamer and the at least one of the first surface and the second surface comprises poly(methyl methacrylate) (PMMA), the aptamer can be immobilized on the PMMA surface by UV activation as described by R. S. Massey and R. Prakash, “A Low-Temperature-Processed, Soft-Fluidic OEGFET Saliva Aptasensor for Cortisol,” in IEEE Journal on Flexible Electronics, vol. 1, no. 1, pp. 64-72, January 2022. In at least one embodiment, when the at least one of the first surface and the second surface comprises polycaprolactone (PCL), the biorecognition entity, including, but not limited to, an aptamer or an antibody, can be bound to the first or second surface using the reagents 1-ethyl-3-(3-dimethylaminopropyl)carbodiimide (EDC) and N-hydroxysuccinimide (NHS) to covalently bond an amine group on the biorecognition entity to the polycaprolactone, using chemical procedures well known in the art and described in, for example, M. Cooper, “Sensor surfaces and receptor deposition,” in Label-free biosensors techniques and applications, M. Cooper Ed. Cambridge, UK: Cambridge University Press, pp. 110-142, 2009. In at least one embodiment, when the at least one of the first surface and the second surface comprises polylactic acid (PLA), whichever of the first surface or the second surface bears the biorecognition entity contains a polylactic acid—polyethylene glycol—carboxylic acid block copolymer. The presence of such a block copolymer bearing carboxyl groups can facilitate covalent binding of a biorecognition entity, including, but not limited to, an aptamer or an antibody, to the first or second surface by crosslinking using the EDC-NHS method described above.

Another aspect of the present application provides a method of producing an organic electrolyte-gated field effect transistor biosensor as described herein. In at least one embodiment, the method includes:
3D printing a thermoplastic dielectric material to form a microfluidic channel structure comprising at least one interior surface;
disposing the microfluidic channel structure between a gate electrode and a semiconductor material in electrical contact with a source electrode and a drain electrode such that the at least one interior surface is separated from the gate electrode and the semiconductor material by the dielectric material; and
binding a biorecognition entity to at least a portion of the at least one interior surface.

In at least one embodiment, the thermoplastic dielectric material is 3D-printed to form a first dielectric layer as described herein between the gate electrode and the at least one interior surface. In at least one embodiment, the thermoplastic dielectric material is 3D-printed to form a second dielectric layer as described herein between the semiconductor material and the at least one interior surface.

In at least one embodiment, the method includes:
depositing a gate electrode onto a first substrate;
3D printing a dielectric material onto a gate electrode to form a first dielectric layer having a first surface;
depositing a source electrode and a drain electrode onto a second substrate;
depositing a semiconductor material on the second substrate so as to electrically connect the source electrode and the drain electrode to the semiconductor material;
3D printing the dielectric material onto the semiconductor material to form a second dielectric layer having a second surface;
binding a biorecognition entity to at least one of the first surface and the second surface and
joining the first dielectric layer to the second dielectric layer with one or more sidewalls, such that the first surface, the second surface and the one or more sidewalls define a microfluidic channel therebetween.

In at least one embodiment, the method includes:
depositing a source electrode and a drain electrode onto a substrate;
depositing a semiconductor material on the substrate so as to connect the source electrode and the drain electrode to the semiconductor material;
3D printing a dielectric material onto the semiconductor material to form a microfluidic channel structure, the microfluidic channel structure comprising a first dielectric layer having a first surface, a second dielectric layer having a second surface and one or more sidewalls between the first and second dielectric layers to define a microfluidic channel between the first surface and the second surface, wherein the second dielectric layer is between the semiconductor material and the microfluidic channel;
binding a biorecognition entity to at least one of the first surface and the second surface; and
depositing a gate electrode onto the first dielectric layer such that the first dielectric layer is between the gate electrode and the microfluidic channel.

In at least one embodiment, the method includes:
depositing a gate electrode onto a substrate;
3D printing a dielectric material onto the gate electrode to form a microfluidic channel structure, the microfluidic channel structure comprising a first dielectric layer having a first surface, a second dielectric layer having a second surface and one or more sidewalls between the first and second dielectric layers to define a microfluidic channel between the first surface and the second surface, wherein the first dielectric layer is between the gate electrode and the microfluidic channel;
binding a biorecognition entity to at least one of the first surface and the second surface;
depositing a semiconductor material on the second dielectric layer, wherein the second dielectric layer is between the semiconductor material and the microfluidic channel.
depositing a source electrode and a drain electrode onto the second dielectric layer so as to electrically connect the source electrode and the drain electrode to the semiconductor material.

In at least one embodiment, the method includes depositing a self-assembled monolayer onto the second substrate or onto the second dielectric layer and depositing an organic or carbon-based semiconductor material onto the self-assembled monolayer. In at least one embodiment, the self-assembled monolayer comprises octadecyltrichlorosilane.

In at least one embodiment, the microfluidic channel structure is printed three-dimensionally from the dielectric material to form a monolithic structure. Preparing the biosensor in this way provides that the first and second dielectric layers defining the microfluidic channel can be prepared with more reproducibility during the 3D printing process and are unitarily formed within the microfluidic channel structure, avoiding the problems associated with molding a separate microfluidic channel from PDMS and crosslinking it to separately formed first and second dielectric layers, as seen in previously known devices.

In another aspect, the present application provides a method of analyzing an electrolyte fluid for an analyte, comprising exposing an organic electrolyte-gated field effect transistor biosensor as described herein to the electrolyte fluid and determining one or more features of the analyte. In at least one embodiment, the electrolyte fluid is a sample obtained from an environmental source. In at least one embodiment, the electrolyte fluid is a sample obtained from an agricultural source. In at least one embodiment, the electrolyte fluid is a sample obtained from a food source. In at least one embodiment, the electrolyte fluid is a sample obtained from a manufacturing process. In at least one embodiment, the electrolyte fluid is a biological fluid. In at least one embodiment, the biological fluid is one or more of blood, plasma, urine, sweat, interstitial fluid, cerebrospinal fluid, exhaled breath condensate or saliva. In at least one embodiment, the electrolyte fluid is saliva. In at least one embodiment, the electrolyte fluid is blood. In at least one embodiment, the electrolyte fluid is plasma. In at least one embodiment, the analyte is a biomarker whose presence or concentration is indicative of a medical condition, as described herein. In at least one embodiment, exposing the biosensor to the electrolyte fluid comprises exposing the microfluidic channel of the biosensor to the electrolyte fluid. In at least one embodiment, determining one or more features of the analyte comprises determining the presence of the analyte in the electrolyte fluid or measuring the concentration of the analyte in the electrolyte fluid. In at least one embodiment, interaction of the analyte with the biorecognition entity results in one or more of formation of a charge separation region at an interface between the electrolyte fluid and at least one of the first dielectric layer and the second dielectric layer, a change in a net gate-to-channel capacitance of the biosensor, and a change in output current of the biosensor wherein the change in output current is indicative of the one or more features of the analyte in the electrolyte fluid.

As used herein and unless otherwise indicated, the terms “a” and “an” are intended to include both plural and singular forms and can be interpreted to mean “one or more”.

As used herein, the terms “about” or “approximately” as applied to a numerical value or range of values are intended to mean that the recited values can vary within an acceptable degree of error for the quantity measured given the nature or precision of the measurements, such that the variation is considered in the art as equivalent to the recited values and provides the same function or result. For example, the degree of error can be indicated by the number of significant figures provided for the measurement, as is understood in the art, and includes but is not limited to a variation of ±1 in the most precise significant figure reported for the measurement. Typical exemplary degrees of error are within 20 percent (%), preferably within 10%, and more preferably within 5% of a given value or range of values. Alternatively, and particularly in biological systems, the terms “about” and “approximately” can mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “substantially” refers to the complete or nearly complete extent or degree of an action, characteristic, property, state, structure, item, or result. For example, an object that is “substantially” in a given position including, but not limited to, vertical, horizontal, or adjacent to or aligned with another object, would mean that the object is either completely in that position or nearly completely in that position. The exact allowable degree of deviation from absolute completeness may, in some cases, depend on the specific context. However, generally speaking the nearness of completion will be so as to have the same overall result as if absolute and total completion were obtained.

The use of “substantially” is equally applicable when used in a negative connotation to refer to the complete or near complete lack of an action, characteristic, property, state, structure, item, or result. For example, a composition that is “substantially free of” an ingredient or element would either completely lack that ingredient or element, or so nearly completely lack that ingredient or element that the effect would be the same as if it completely lacked that ingredient or element. In other words, a composition that is “substantially free of” an ingredient or element may still actually contain such item as long as there is no measurable or significant effect thereof.

As used herein, terms indicating relative direction or orientation, including, but not limited to, “upper”, “lower”, “top”, “bottom”, “vertical”, “horizontal”, “outer”, “inner”, “front”, “back”, and the like, are intended to facilitate description of the present invention by indicating relative orientation or direction in usual use, and are not intended to limit the scope of the present invention in any way to such orientations or directions.

### EXAMPLES

Other features of the present invention will become apparent from the following non-limiting examples which illustrate, by way of example, the principles of the invention.

### Example 1: General Procedure for Preparation of Biosensors

Embodiments of the present biosensor can be prepared using the following general procedure. A gate electrode is printed on a flexible Kapton™ substrate with silver nanoparticle ink, using screen printing or extrusion printing technology. A solution of 5 wt % polycaprolactone (PCL) in chloroform is deposited on the gate electrode by extrusion printing, and dried at 30° C. The microfluidic channel structure can be formed by 3D printing of PCL or polylactic acid (PLA) on the PCL surface. In embodiments containing PLA, the PCL surface is activated using 0.1 M NaOH and PLA is deposited on a portion of the PCL surface by 3D printing from melt (200° C.) to form the microfluidic channel, leaving a portion of the PCL surface exposed.

Biorecognition entities are then covalently bonded to the exposed PCL surface using the reagents 1-ethyl-3-(3-dimethylaminopropyl)carbodiimide (EDC) and N-hydroxysuccinimide (NHS) as described in, for example, M. Cooper, “Sensor surfaces and receptor deposition,” in Label-free biosensors techniques and applications, M. Cooper Ed. Cambridge, UK: Cambridge University Press, pp. 110-142, 2009. Alternatively, a coating of poly(methyl methacrylate) (PMMA) may be deposited on the PCL surface prior to covalently bonding a biorecognition entity to the PMMA coating. In at least one embodiment, an aptamer can be bonded to the PMMA coating by drop casting with UV activation, as described in R. S. Massey and R. Prakash, IEEE Journal on Flexible Electronics (2022), 1(1): 64-72. Antibodies can also be covalently bound to the PMMA using the EDC-NHS procedure described above.

Source and drain electrodes are printed on a flexible Kapton™ substrate with silver nanoparticle ink, again using screen printing or extrusion printing technology. Semiconductor material is deposited on the flexible substrate as described in Example 2 below and annealed so as to connect the source and drain electrodes. A solution of 9 wt % PLA in 1,4-dioxane or of 5 wt % PCL in chloroform is deposited on the semiconductor material by extrusion printing and contacted with the exposed surface of the 3D printed PLA or PCL microchannel described above while still wet to seal the microfluidic channel.

Alternatively, a trilayer dielectric layer can be formed on the semiconductor material. A layer of PLA on the semiconductor material is deposited on the semiconductor material by spin coating using a 2 mg/mL solution of PLA in chloroform, followed immediately by spin coating the PLA layer with an 80 mg/mL solution of polyvinyl alcohol (PVA) containing 0.75 wt. % of cellulose nanocrystals. The bilayer of PLA and PVA-cellulose is annealed for 1 h under vacuum at 150° C. and a 2 mg/mL solution of toluene diisocyanate terminated polycaprolactone (TPCL) is spin coated onto the PVA-cellulose layer and the trilayer is annealed for 15 minutes under vacuum at 200° C.

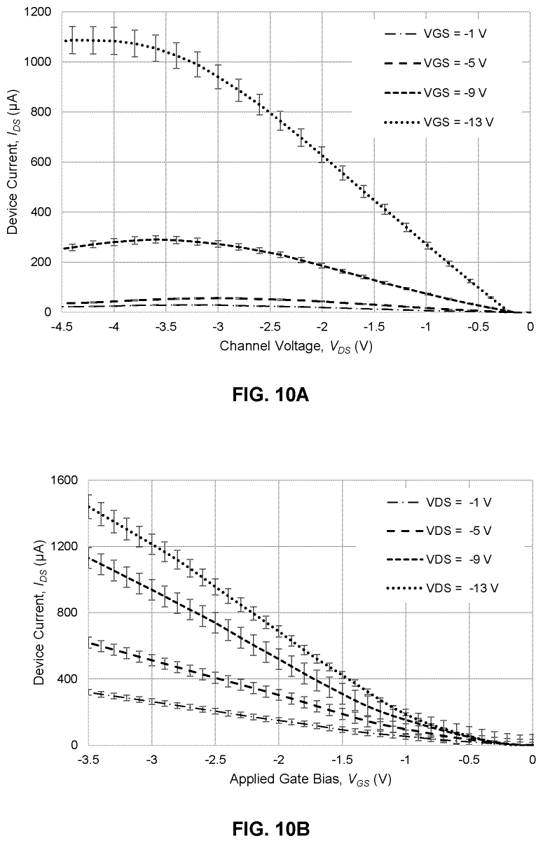

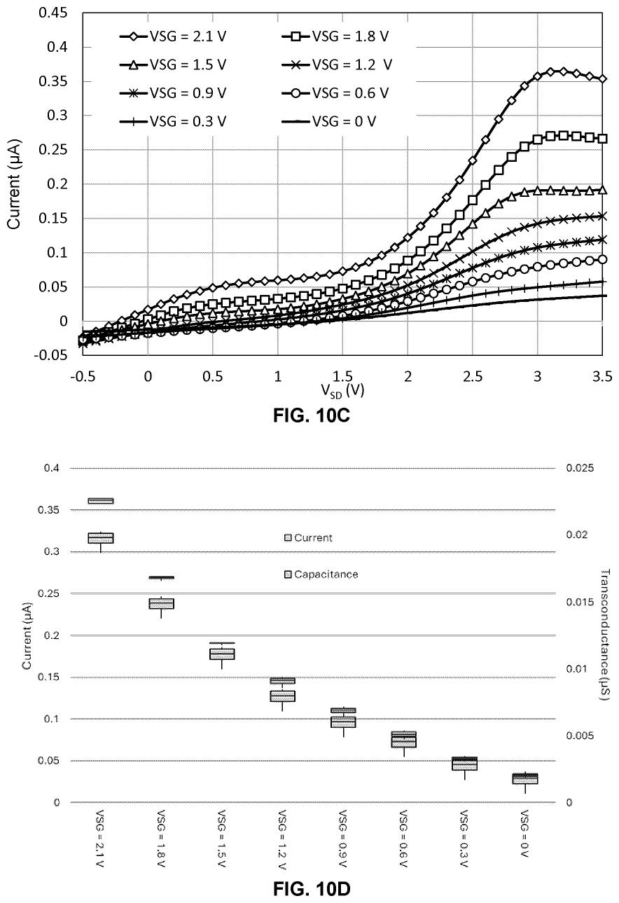

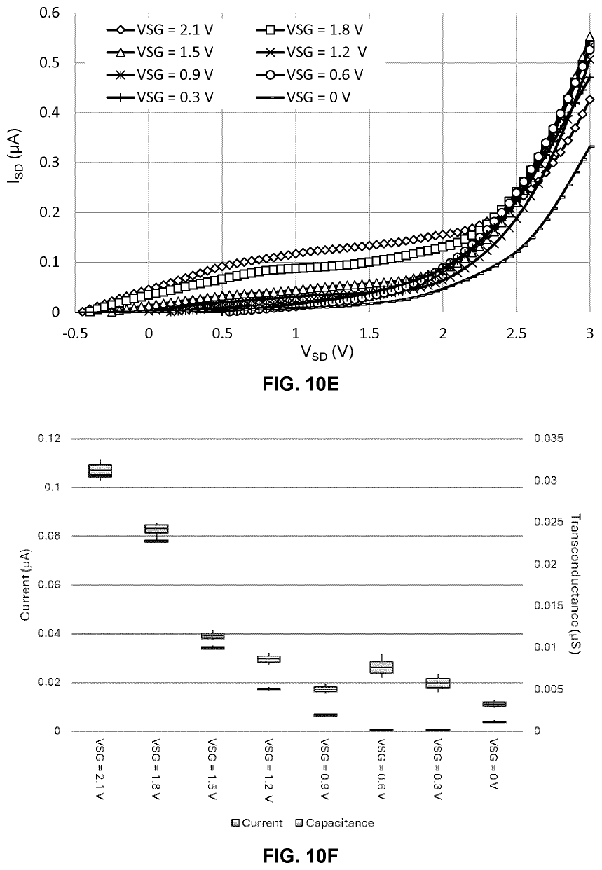

Embodiments of the present biosensor prepared according to the general procedure outlined above were tested for transistor function with deionized water as the electrolyte in the microfluidic channel using a general procedure as is well known in the art. Varying voltages are applied to the source, drain and gate electrodes and the device output currents and device transfer function responses (output current modulated by gate bias voltage) are measured using standard laboratory equipment such as semiconductor parameter analyzers or a bespoke Systems-on-Board hybrid integrated system for low power, portable analysis. Representative graphs obtained for embodiments of the present biosensor are shown in FIGS. 10A to 10F, respectively.

### Example 2: Deposition of Semiconductor Material

Various embodiments of the present biosensor were prepared using the general procedure of Example 1 but using alternative methods of depositing the semiconductor material on the source and drain electrodes. Embodiments are prepared by depositing the semiconductor material TIPS-pentacene by drop-casting, spin-depositing, screen printing or inkjet printing (piezoelectric drop-on-demand (DoD)) of a 1 wt % solution of TIPS-pentacene in chlorobenzene or by inkjet printing of a 1 wt % solution of TIPS-pentacene in 15% chloroform and 84% chlorobenzene by weight.

Alternatively, a semiconductor material including carbon nanotubes can be deposited on the flexible substrate. A mixture of commercially available carbon nanotubes and poly(9,9′-didodecylfluorene-co-N-(2′-decyltetradecane)-carbazole (PCPF) (Mn=65, PD=2.7) dispersed in toluene is purified as described by Rice, N. et al, Advanced Electronic Materials (2019), 5(1): 1800539 and deposited using drop-casting or inkjet/aerosol jet printing.

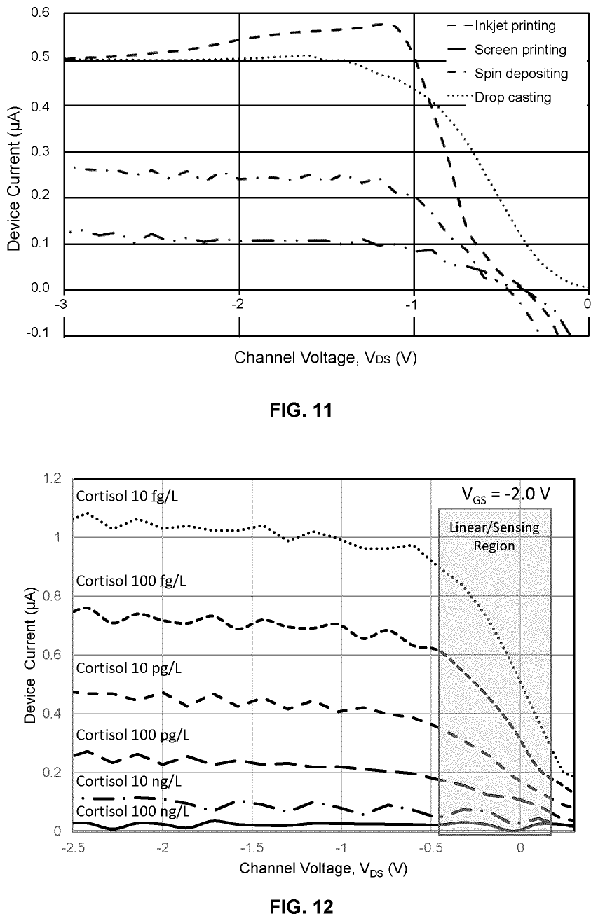

The device output curves of alternative embodiments prepared by inkjet printing from a 1 wt % solution of TIPS-pentacene in 15% chloroform and 84% chlorobenzene, or by screen printing, spin depositing and drop casting of the semiconductor material from a 1 wt % solution of TIPS-pentacene in chlorobenzene are shown in FIG. 11. As can be seen from the data presented in FIG. 11, inkjet printing of the semiconductor material from a 1 wt % solution of TIPS-pentacene in 15% chloroform and 84% chlorobenzene provided a device with the most favourable features. It is contemplated that this method can offer precision deposition and controlled device geometries which will improve device yield and lower the variation between batches.

### Example 3: Testing for Detection of Analytes

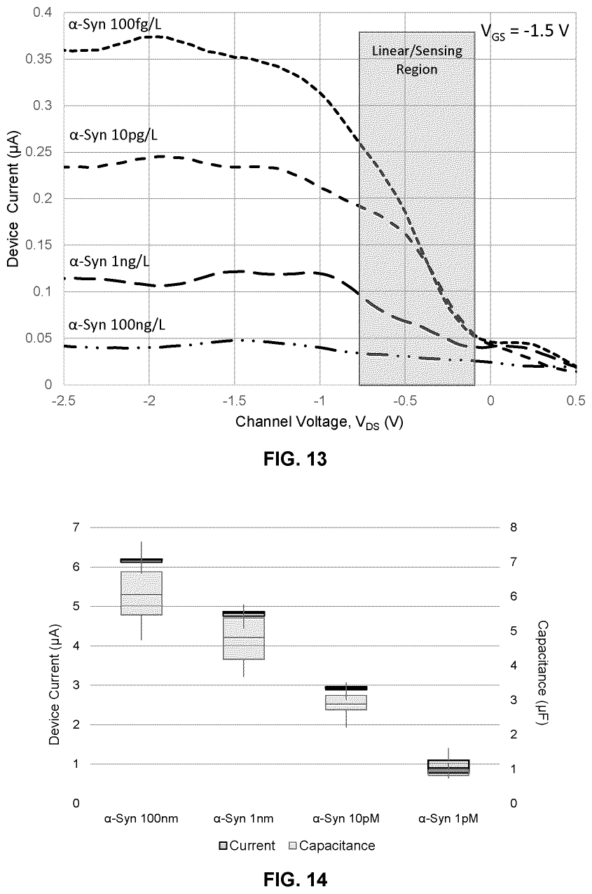

Embodiments of the present biosensor device were prepared according to the procedures of Examples 1 and 2 including both PCL and PLA as dielectric material and bearing aptamers recognizing either cortisol or α-synuclein. FIGS. 12 to 14 show device characteristics measured in the presence of a buffer containing various concentrations of cortisol or α-synuclein or in the presence of saliva spiked with various concentrations of α-synuclein, as described in Massey, R. S. et al, “Non-invasive Monitoring of Alpha-synuclein in Saliva for Parkinson's Disease using Organic Electrolyte Gated FET Aptasensor”, ACS Sensors (2023), 8(8): 3116-3126. It can be seen from the results shown in FIGS. 12 to 14 that the present devices show characteristics allowing them to perform as biosensors.

### Example 4: Biosensors Containing Carbon Nanotube Semiconductor Material

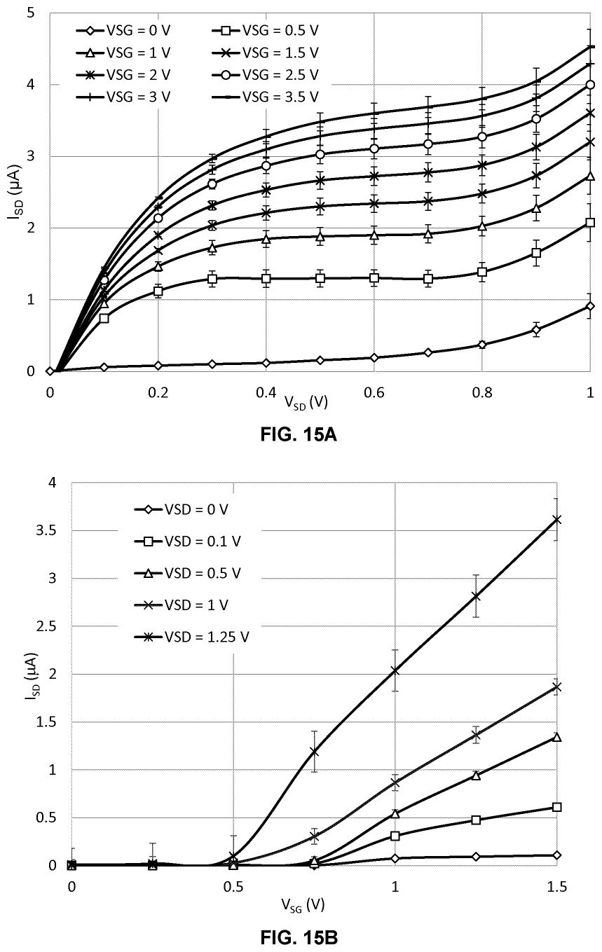

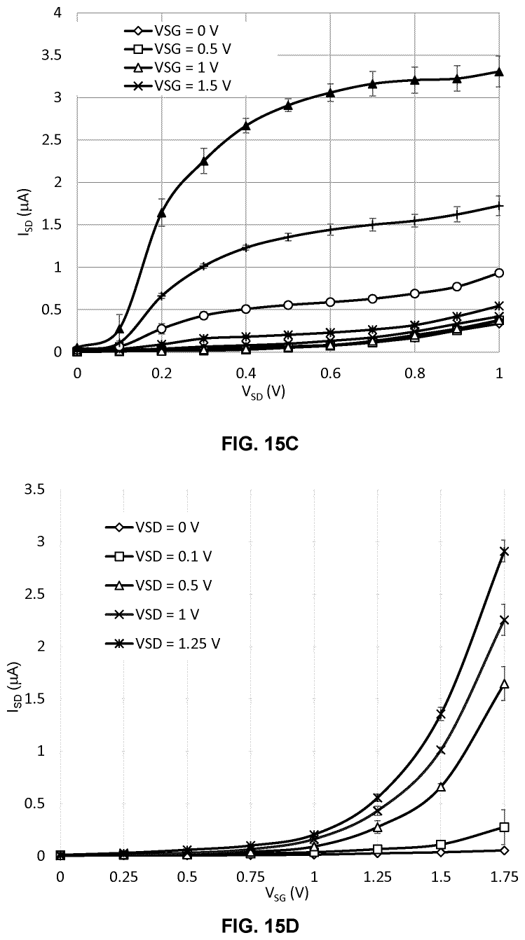

Embodiments of the present biosensor device in which the semiconductor material includes single-walled carbon nanotubes were prepared as described in Examples 1 and 2. In at least one such embodiment, the dielectric material deposited as the second dielectric layer between the semiconductor material and the PCL microfluidic channel structure is a trilayer dielectric material comprising PLA, PVA containing cellulose nanocrystals and thermally crosslinked PCL as described herein. FIGS. 15A to 15D show the device characteristics of such an embodiment measured when the microfluidic channel contains deionized water (FIGS. 15A and 15B) or commercially available Tris (tris(hydroxymethyl)-aminomethane) acetate EDTA (ethylenediaminetetraacetic acid) (TAE) buffer (FIGS. 15C and 15D). As can be seen from FIGS. 15A to 15C, the transconductance and channel current of the device at a given applied gate voltage increase as the conductivity of the electrolyte increases from that of deionized water to that of the buffer solution.

### Example 5: Biosensors Bearing Antibodies as Biorecognition Entities

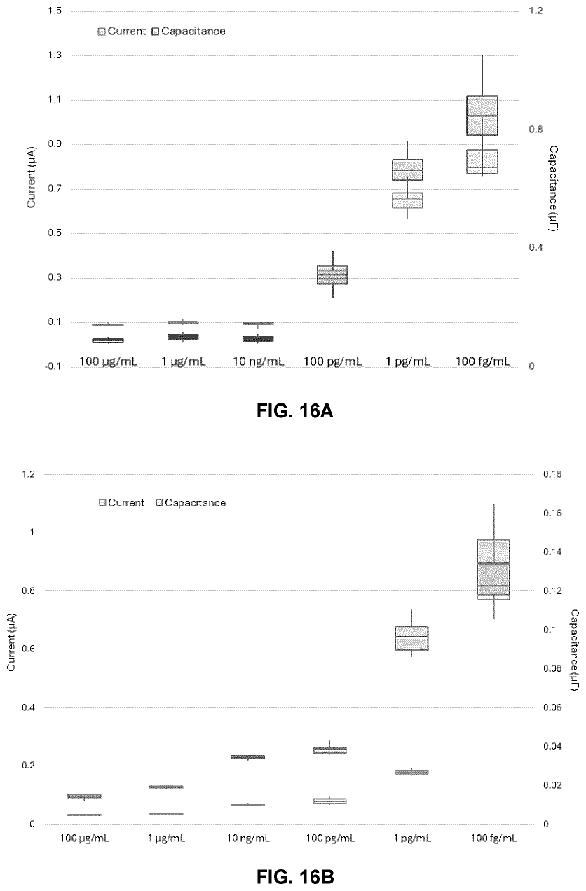

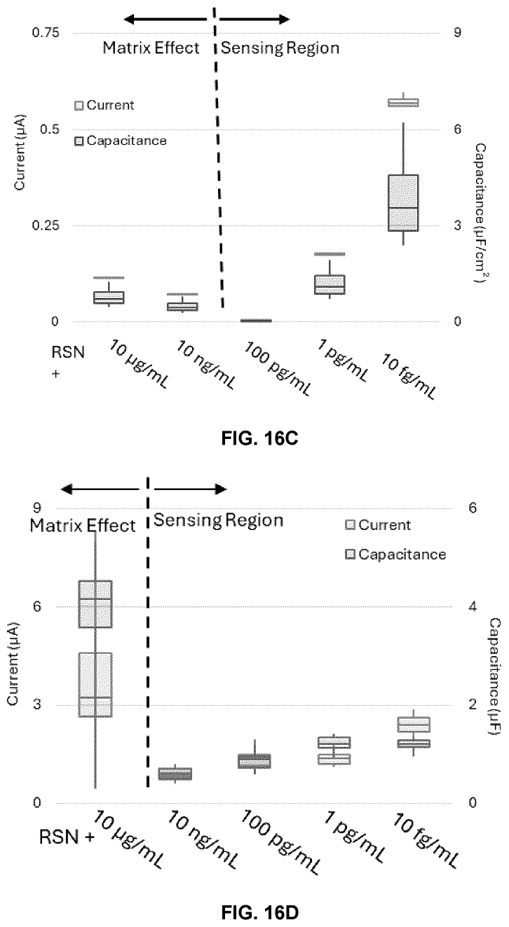

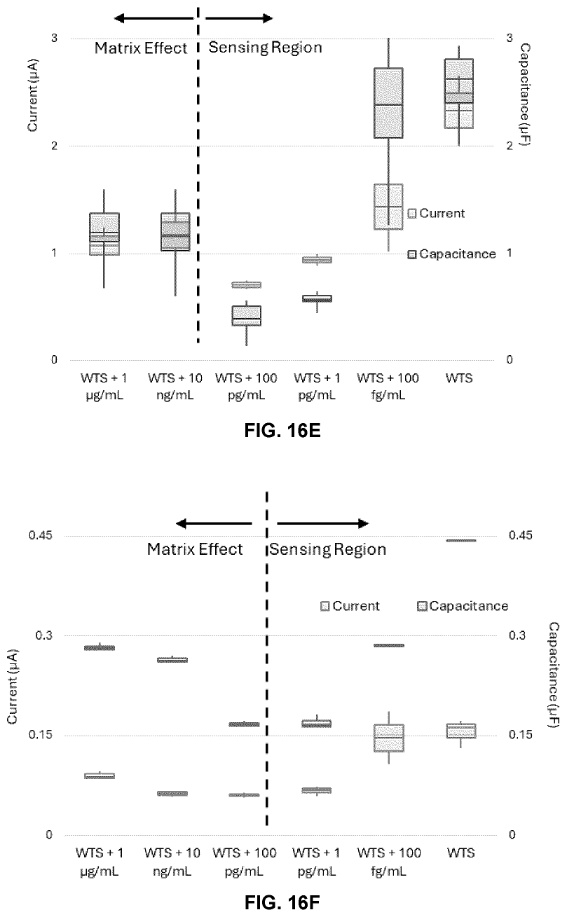

Embodiments of biosensor devices prepared as described in Examples 1 and 2 and bearing either an aptamer recognizing α-synuclein or an antibody recognizing amyloid β-42 as a biorecognition entity were tested for detection of the corresponding analyte (α-synuclein or amyloid β-42, respectively) as described in Massey, R. S. et al, ACS Sensors (2023), 8(8): 3116-3126, with the exception that serum samples are diluted 1:1 with phosphate-buffered saline (PBS) instead of deionized water. As seen from the data shown in FIGS. 16A-F, comparable concentration-dependent detection behaviour was exhibited by the devices biofunctionalized with the antibody or with the aptamer when the respective analyte was present in TAE buffer (FIGS. 16A and 16B), in real saliva supernatant samples (FIGS. 16C and 16D) and in real blood serum samples (FIGS. 16E and 16F). The aptamer-based sensor showed slight differences from the antibody-based sensor in the limit of detection (LOD) and matrix effect. Without being bound by theory, it is contemplated that these differences may be attributable, at least in part, to the larger size of antibodies compared to aptamers in general, resulting in differences in Debye length at the biofunctionalized surface and differences in the rapidity at which binding of the sensor area to analyte is saturated.

### Example 6: Comparison with Previously Known Devices

Embodiments A to D of the present biosensor were prepared according to Examples 1 and 2 above. Embodiments A and C were prepared using only PCL to prepare the microfluidic channel structure, while the microfluidic channel structure of Embodiment B contains both PCL and PLA and the microfluidic channel structure of Embodiment D contains PCL in the first dielectric layer between the gate and the microfluidic channel and a trilayer dielectric layer containing PLA, PVA containing cellulose nanocrystals and thermally crosslinked PCL, as described herein, in the second dielectric layer between the semiconductor material and the microfluidic channel. Embodiments A and B contain TIPS-pentacene as the semiconductor material and Embodiments C and D contain carbon nanotubes as the semiconductor material. Comparative Devices 1 and 2 were prepared as previously described (R. S. Massey and R. Prakash, “A Low-Temperature-Processed, Soft-Fluidic OEGFET Saliva Aptasensor for Cortisol,” in IEEE Journal on Flexible Electronics, vol. 1, no. 1, pp. 64-72, January 2022; Massey, R. S.; Prakash, R. Modeling the Double Layer Capacitance Effect in Electrolyte Gated FETs with Gel and Aqueous Electrolytes. Micromachines (2021), 12, 1569, 1-15).

The characteristics of Comparative Devices 1 and 2, and of Embodiments A to D, were measured using standard procedures known in the art, and the results are shown in Table 1 below. “W/L” represents the width-to-length ratio or aspect ratio of the device.

TABLE 1

| | Device Architecture | | | | | Gate | |
|---|---|---|---|---|---|---|---|
| | | Device | Microfluidic | Capacitance | Threshold | Leakage | Device |
| | | Area | channel | Density, ĉ_{di} | voltage, V_{T} | Current | On/Off |
| Device | W/L | (μm²) | depth (μm) | (μF/cm²) | (V) | (nA) | ratio |
| Comparative | 50 | 1000 | 400 | 1.4 | −1.2 | 0.2-36.7 | 5.5 × 10²- |
| Device 1 | | | | | | | 1.1 × 10⁴ |
| (PVA/PMMA) | | | | | | | |
| Comparative | 50 | 1000 | 375 | 1.2 | −1.1 | 10-52.8 | 9.8 × 10²- |
| Device 2 | | | | | | | 3.2 × 10⁴ |
| (PDMS/PMMA) | | | | | | | |
| Present device | 50 | 1000 | 550 | 2.8 | −1.8 | 0.2-1.5 | 10 × 10⁴- |
| Embodiment A | | | | | | | 2.2 × 10⁶ |
| (PCL/PCL) | | | | | | | |
| Present device | 50 | 1000 | 600 | 2.6 | −1.6 | 0.5-1.2 | 5 × 10⁴- |
| Embodiment B | | | | | | | 3 × 10⁶ |
| (PLA/PCL) | | | | | | | |
| Present device | 50 | 1000 | 300 | 0.99 ± 1.35 | 0.5 | 10-80 | 10²-10⁴ |
| Embodiment C | | | | | | | |
| (PCL/PCL) | | | | | | | |
| Present device | 50 | 1000 | 300 | 2.17 ± 2.1 | 0.5 | 8-60 | 10²-10³ |
| Embodiment D | | | | | | | |
| (PCL/trilayer) | | | | | | | |

As seen from the results presented in Table 1, Embodiments A and B show lower gate leakage current and a higher on/off ratio compared to the Comparative Devices 1 and 2, which would be expected to result in an improvement in sensitivity and shelf life of the present biosensors. In addition, Embodiments C and D show at least comparable performance to the Comparative Devices 1 and 2, but show better protection of the semiconductor layer from moisture and environmental doping, and less variation and sensor drift between batches.

The embodiments described herein are intended to be illustrative of the present compositions and methods and are not intended to limit the scope of the present invention. Various modifications and changes consistent with the description as a whole and which are readily apparent to the person of skill in the art are intended to be included. The appended claims should not be limited by the specific embodiments set forth in the examples but should be given the broadest interpretation consistent with the description as a whole.

1-21. (canceled)

22. An organic electrolyte-gated field effect transistor biosensor comprising:
a gate electrode;
a source electrode;
a drain electrode;
a semiconductor material in electrical contact with the source electrode and the drain electrode; and
a microfluidic channel structure comprising at least one thermoplastic dielectric material and at least one interior surface bearing a biorecognition entity, wherein the at least one interior surface is separated from the gate electrode, the source electrode, the drain electrode and the semiconductor material by the thermoplastic dielectric material and wherein the thermoplastic dielectric material comprises one or more materials selected from the group consisting of polylactic acid (PLA), polycaprolactone (PCL), poly(ethyl methacrylate) (PEMA), polybutylene terephthalate (PBT), polyethylene terephthalate (PET), polyethylene (PE), polypropylene (PP), polystyrene (PS), polytetrafluoroethylene (PTFE), cellulose, acylated cellulose derivatives, cellulose acetate, cellulose acetate butyrate, cellulose propionate, acrylonitrile butadiene styrene (ABS), acrylonitrile styrene acrylate (ASA), polycaprolactam (PC), polybutadiene (PBD), polydimethylsiloxane (PDMS) and nylon polyamides.

23. The organic electrolyte-gated field effect transistor biosensor of claim 22, wherein the thermoplastic dielectric material comprises at least one of polylactic acid (PLA) and polycaprolactone (PCL).

24. The organic electrolyte-gated field effect transistor biosensor of claim 22, wherein the microfluidic channel structure comprises:
a first dielectric layer having a first surface;
a second dielectric layer having a second surface parallel to and opposing the first surface; and
one or more sidewalls contiguous with the first dielectric layer and the second dielectric layer so as to separate the first dielectric layer from the second dielectric layer,
wherein the first surface, the second surface and the one or more sidewalls define a microfluidic channel therebetween, wherein the microfluidic channel is configured to receive an electrolyte fluid such that the electrolyte fluid is in contact with the first surface and the second surface, and
wherein at least one of the first surface and the second surface bears the biorecognition entity.

25. The organic electrolyte-gated field effect transistor biosensor of claim 24, wherein one or both of the first dielectric layer and the second dielectric layer comprise at least one of polylactic acid (PLA) and polycaprolactone (PCL).

26. The organic electrolyte-gated field effect transistor biosensor of claim 24, wherein at least one of the first dielectric layer and the second dielectric layer comprises a layer comprising PLA, a layer comprising polyvinyl alcohol (PVA) and nanocrystalline cellulose, and a layer comprising PCL.

27. The organic electrolyte-gated field effect transistor biosensor of claim 22, wherein the semiconductor material is an organic or carbon-based semiconductor material.

28. The organic electrolyte-gated field effect transistor biosensor of claim 27, wherein the organic or carbon-based semiconductor material is selected from the group consisting of 6,13-bis(triisopropylsilylethynyl)pentacene (TIPS-pentacene), polythiophenes and copolymers thereof, metal phthalocyanines, and single-walled carbon nanotubes.

29. The organic electrolyte-gated field effect transistor biosensor of claim 28, wherein the organic or carbon-based semiconductor material is TIPS-pentacene or single-walled carbon nanotubes.

30. The organic electrolyte-gated field effect transistor biosensor of claim 27, further comprising a self-assembled monolayer, wherein the organic or carbon-based semiconductor material is deposited on the self-assembled monolayer.

31. The organic electrolyte-gated field effect transistor biosensor of claim 22, wherein the biorecognition entity is an aptamer or an antibody.

32. A method of analyzing an electrolyte fluid for an analyte, the method comprising exposing the organic electrolyte-gated field effect transistor biosensor of claim 22 to the electrolyte fluid and determining one or more features of the analyte in the electrolyte fluid.

33. The method of claim 32, wherein the electrolyte fluid is a biological fluid selected from blood, plasma, urine, sweat, interstitial fluid, cerebrospinal fluid, saliva and exhaled breath condensate.

34. The method of claim 33, wherein the electrolyte fluid is selected from blood, plasma, and saliva.

35. The method of claim 32, wherein the one or more features of the analyte in the electrolyte fluid comprise the presence of the analyte in the electrolyte fluid or the concentration of the analyte in the electrolyte fluid.

36. The method of claim 32, wherein the analyte in the electrolyte fluid is selected from the group consisting of drugs, metabolites, hormones, neurotransmitters, enzymes, carcinogens, peptides, proteins, electrolytes, metal ions, nucleic acids and cells.

37. The method of claim 32, wherein the analyte in the electrolyte fluid is an analyte useful for measuring neural function, cardiac function, liver function or kidney function or for detecting neurodegenerative conditions, cardiac conditions or disorders, blood conditions or disorders, infection, conditions related to inflammation or stress, hepatic conditions or disorders, renal conditions or disorders and cancer or precancerous conditions.

38. The method of claim 37, wherein the analyte in the electrolyte fluid is selected from the group consisting of α-synuclein, β-amyloids, and tau proteins.

39. The method of claim 32, wherein interaction of the analyte with the biorecognition entity results in one or more of formation of a charge separation region at an interface between the electrolyte fluid and at least one of the first dielectric layer and the second dielectric layer, a change in a net gate-to-channel capacitance of the biosensor, and a change in output current of the biosensor wherein the change in output current is indicative of the one or more features of the analyte in the electrolyte fluid.

40. A method of producing an organic electrolyte-gated field effect transistor biosensor according to claim 22, the method comprising:
3D printing a thermoplastic dielectric material to form a microfluidic channel structure comprising at least one interior surface;
disposing the microfluidic channel structure between a gate electrode and a semiconductor material in electrical contact with a source electrode and a drain electrode such that the at least one interior surface is separated from the gate electrode and the semiconductor material by the dielectric material; and
binding a biorecognition entity to at least a portion of the at least one interior surface.

41. The method of claim 40, wherein the microfluidic channel structure is printed three-dimensionally from the thermoplastic dielectric material as a monolithic structure.
